SEQUENCE LISTING (8 sequences)

SEQ ID NO: 1 - DNA, 23 bp, Artificial Sequence
  Description of Artificial Sequence Synthetic primer
gatggtgaagatggtcccacagg

SEQ ID NO: 2 - DNA, 22 bp, Artificial Sequence
  Description of Artificial Sequence Synthetic primer
ggtcgtccgggttttccagggt

SEQ ID NO: 3 - DNA, 24 bp, Artificial Sequence
  Description of Artificial Sequence Synthetic probe
ttccaaggacctgctggtgagcct

SEQ ID NO: 4 - DNA, 26 bp, Artificial Sequence
  Description of Artificial Sequence Synthetic probe
tgaacctggtcaaactggtcctgcag

SEQ ID NO: 5 - DNA, 29 bp, Artificial Sequence
  Description of Artificial Sequence Synthetic oligonucleotide
cccttggtgggggcggggcctaagctgcg

SEQ ID NO: 6 - DNA, 29 bp, Artificial Sequence
  Description of Artificial Sequence Synthetic oligonucleotide
cccttggtgggttgggggcctaagctgcg

SEQ ID NO: 7 - DNA, 20 bp, Artificial Sequence
  Description of Artificial Sequence Synthetic oligonucleotide
cgcaggctcctcccagctgt

SEQ ID NO: 8 - DNA, 20 bp, Artificial Sequence
  Description of Artificial Sequence Synthetic oligonucleotide
cgcaggcgaatcccagctgt

(19) United States
(12) Patent Application Publication
(10) Pub. No.: US 2006/0009452 A1
(43) Pub. Date: Jan. 12, 2006

(54) Therapeutic targeting of PARC/CCL18 and its signaling in pulmonary fibrosis

(76) Inventors: Sergei P. Atamas, Columbia, MD (US); Irina G. Luzina, Columbia, MD (US); Barbara White, Finksburg, MD (US)

Correspondence Address: CASTELLANO MALM FERRARIO & BUCK PLLC, 2121 K STREET, NW, SUITE 800, WASHINGTON, DC, 20037, US

(21) Appl. No.: 11/143,707

(22) Filed: Jun. 3, 2005

Related U.S. Application Data
(60) Provisional application No. 60/576,442, filed on Jun. 3, 2004.

(51) Int. Cl. A61K 31/5377

(52) U.S. Cl. 514/232.8

(57) ABSTRACT

The present invention relates to methods of treating, preventing or preventing the progression of fibrosis comprising inhibiting the actions of pulmonary and activation-regulated chemokine (PARC) or at least one of its downstream effector molecules, such as Sp1 transcription factor and protein kinase C-alpha (PKCα). The present invention also relates to methods of screening and/or identifying compounds useful for the treatment of fibrosis comprising contacting PARC or its downstream effector molecules, such as Sp1 or PKCα, with a substance and subsequently determining the effects of the substance on the activity of PARC or Sp1 or PKCα. The present invention also relates to methods of screening and/or identifying compounds that prevent or inhibit collagen deposition comprising contacting PARC or its downstream effector molecules, such as Sp1 or PKCα, with a substance and subsequently determining the effects of the substance on the activity of PARC or Sp1 or PKCα.

### CROSS-REFERENCE TO RELATED APPLICATIONS

This application claims priority to U.S. provisional application Ser. No. 60/576,442, filed Jun. 3, 2004, the entirety of which is hereby incorporated by reference.

### STATEMENT REGARDING FEDERALLY SPONSORED RESEARCH OR DEVELOPMENT

Part of the work performed during development of this invention utilized U.S. Government funds from NIH Grant No. 1R01HL074067. The U.S. Government has certain rights in this invention.

### BACKGROUND OF THE INVENTION

1. Field of the Invention

The present invention relates to methods of preventing, treating or preventing the progression of fibrosis in a subject, comprising modulating the activity or expression of a CC chemokine CCL18, also known as pulmonary activation-regulated chemokine (PARC), and/or its effector molecules. Methods of identifying compounds that modulate the activation or expression of PARC/CCL18 and/or its effector molecules are also disclosed.

2. Background of the Invention

Pulmonary fibrosis is a major cause of death in scleroderma patients. Restrictive lung disease develops in 30-60% of patients with systemic sclerosis (scleroderma) within the first three to five years of disease and progresses to severe restrictive lung disease in about 15% of patients.

Pulmonary fibrosis can cause decreased oxygen in the blood (hypoxia), which can, in turn, lead to elevated pressure in the pulmonary artery (pulmonary hypertension), subsequently leading to right ventricular failure. Therefore, patients with pulmonary fibrosis are often treated with supplemental oxygen to prevent pulmonary hypertension.

The mechanisms that lead to progressive lung fibrosis in scleroderma remain obscure. The immune system, however, is thought to play a central role in the development of most forms of pulmonary fibrosis. For example, lung inflammation is present in a subset of scleroderma patients and is associated with a greater risk of acquiring progressive lung fibrosis and death. Compared to patients without lung inflammation, a variety of cell types, including alveolar macrophages, CD8+ T-cells, mast cells, basophils, eosinophils, and neutrophils, are increased in bronchoalveolar lavage (BAL) fluids in scleroderma patients with accompanying lung inflammation. And inflammatory mediators, such as thrombin, fibronectin, transforming growth factor-β (TGF-β), endothelin-1, and type 2 cytokine, are reportedly increased in BAL fluids or cells taken from patients with scleroderma.

The treatment of idiopathic pulmonary fibrosis frequently involves corticosteroids, such as prednisone, and/or other medications that suppress the body's immune system. The goal of current treatment regimens is to decrease lung inflammation and subsequent scarring.

Responses to currently available treatments are variable, and the toxicity and side effects associated with these treatments can be serious. Indeed, only a minority of patients responds to corticosteroids alone, and immune suppression medications are often used in combination with corticosteroids. Such immune suppressive medications used in combination with steroids include, but are not limited to, cyclophosphamide, azathioprine, methotrexate, penicillamine, and cyclosporine. In addition, the anti-inflammatory medication, colchicine, has also been used with some success.

On the other hand, TGF-β is considered to be the central profibrotic cytokine, but is not a good target for the treatment of fibrosis because of its ubiquitous and systemic regulatory effects on the immune system and in connective tissue.

Accordingly, new, more specific treatment and prevention methods of fibrosis are needed in the art.

### SUMMARY OF THE INVENTION

The present invention relates to methods of treating, preventing or preventing the progression of fibrosis comprising inhibiting the actions of pulmonary and activation-regulated chemokine (PARC) or at least one of its downstream effector molecules, such as Sp1 transcription factor and protein kinase C-alpha (PKCα).

The present invention also relates to methods of screening and/or identifying compounds useful for treating, preventing or preventing the progression of fibrosis comprising contacting PARC or its downstream effector molecules, such as Sp1 or PKCα, with a substance and subsequently determining the effects of the substance on the activity of PARC or Sp1 or PKCα.

The present invention also relates to methods of screening and/or identifying compounds that prevent or inhibit collagen deposition comprising contacting PARC or its downstream effector molecules, such as Sp1 or PKCα, with a substance and subsequently determining the effects of the substance on the activity of PARC or Sp1 or PKCα.

### BRIEF DESCRIPTION OF THE DRAWINGS

FIG. 1. A depiction of PARC promotion of fibrosis, directly and indirectly.

FIG. 2. Collagen production after activation of fibroblast cell cultures with recombinant human PARC (rhPARC) for 48 h. (A) Collagen was metabolically labeled with 14C-proline in LF1 cells, culture supernatant separated in PAGE under reducing conditions, and collagen chains visualized by fluorographically enhanced autoradiography. Equal loading was ensured by adjusting total protein in the loaded sample. Samples were loaded as follows: lane 1, control non-stimulated fibroblast supernatant; lane 2, supernatant from fibroblast culture activated with 300 ng/ml rhPARC; and lane 3, sample 2 digested with collagenase. The combined density of procollagen bands in lane 2 is 3.4-fold higher than in lane 1, after adjustment to the local background. (B) Collagen was metabolically labeled with 14C-proline in LF2 cells, fibroblast culture supernatants subjected to PAGE under reducing conditions, and bands visualized as outlined above in A. Samples were loaded as follows: lane 1, control non-stimulated fibroblast culture supernatant; lane 2, supernatant from fibroblasts activated with 30 ng/ml rhPARC; lane 3, supernatant from fibroblasts activated with 300 ng/ml rhPARC; and lane 4, supernatant from fibroblasts activated with 10 ng/ml rhIL-4. The combined density of procollagen bands in lanes 2, 3, and 4 are 2.7-, 4.4-, and 2.9-fold higher, respectively, than in lane 1, after adjustment to the local background. (C) Western blotting of LF2 cell culture supernatants for collagen α1(I). Samples were loaded in the following order: lane 1, human type I collagen (Southern Biotech); lane 2, control non-stimulated fibroblast supernatant; lane 3, supernatant from fibroblasts activated with 300 ng/ml rhPARC; lanes 4-6, sample 3 digested with 125 μg/ml, 25 μg/ml, and 2.5 μg/ml pepsin, respectively. The combined density of procollagen bands in lane 3 is 4.2-fold higher than in lane 2, after adjustment to the local background. (D) Western blotting of LF4 cell culture supernatant for collagen α1(I). Samples were loaded as follows: lane 1, control non-stimulated fibroblast supernatant; lane 2, supernatant from fibroblasts activated with 100 ng/ml rhPARC; lane 3, same as sample 2 incubated with 100 μg/ml neutralizing anti-PARC antibodies. The combined density of procollagen bands in lanes 2 and 3 are 3.6- and 1.4-fold higher, respectively, than in lane 1, after adjustment to the local background.

FIG. 3. Increase in production of collagen depends on the dose of PARC and time of activation. (A and B) Western blotting of LF4 cell culture supernatants for collagen α1(I) (upper part of each panel) and densitometry of the combined pro-α1(I) collagen bands adjusted to local background (lower part of each panel). (A) Fibroblasts were activated for 48 h with increasing concentration of rhPARC from 1 ng/ml to 300 ng/ml. (B) Fibroblast cultures were incubated for 24, 48, and 72 h without (Ctrl) or with 300 ng/ml rhPARC (PARC). The increase in densities of the collagen bands relative to the control culture was 4.4 at 24 h, 3.2 at 48 h, and 1.3 at 72 h, although densities of both pro-α1(1) collagen bands in the PARC-treated sample at 72 h of activation were saturated and underrepresented the true increase in collagen levels. Densitometry of the same bands measured at a shorter, non-saturating exposure, showed a 3.4-fold increase in collagen production by rhPARC-stimulated fibroblasts at 72 h.

FIG. 4. Real-time PCR of 18S rRNA and collagen mRNA in lung fibroblasts, control and treated with 300 ng/ml of rhPARC. Detection of 18S PCR product was done with SYBR Green (fluorescence 1, F11 on the left vertical axis in A) and detection of collagen PCR product was done with specific HybProbes (fluorescence 1/fluorescence 2, F11/F12 on the right vertical axis in (A). (A) Equal RNA concentration in control and PARC-treated cells, based on close overlap of corresponding 18S rRNA amplification curves. This panel also shows that after 6 h of incubation in triplicates, amplification of collagen PCR product from fibroblasts treated with rhPARC occurs approximately two cycles earlier than in control samples, indicating approximately 4-fold higher concentration of collagen mRNA in the treated samples. (B) Kinetics of collagen mRNA increase in LF1 (open bars) and LF2 (shaded bars) fibroblasts treated with PARC versus non-stimulated cells incubated for the same periods of time, after normalization to 18S rRNA. Levels of collagen at 3 and 6 h of activation are higher than in control cells (P<0.05 by one-way ANOVA with post hoc testing), and the differences approach statistical significance at 24 h (P<0.09).

FIG. 5. COLIA2 expression in the presence of PARC versus rhTGF-β, using a COLIA2 reporter assay with CAT-reporter plasmids containing 3500 base pairs fragment of the collagen promoter transiently transfected into human fibroblasts, 48 hours activation, 300 ng/ml rhPARC, and 5 ng/ml rhTGF-β.

FIG. 6. Western blotting of whole cell lysates with anti-phospho-ERK1/2 (A), anti-phospho-p38 (B), and ERK2 for loading control (C), after activating lung fibroblasts LF1 with rhPARC for indicated times (min). Phosphorylation of ERK1/2 but not p38 is activated by rhPARC.

FIG. 7. Transfection of lung fibroblasts with ERK DNM blocks ERK phosphorylation and collagen production in non-stimulated and PARC-stimulated cells. Vertical lanes in all panels correspond to the following fibroblast cultures—1: mock-transfected non-stimulated; 2: mock-transfected PARC-stimulated; 3: ERK DNM-transfected non-stimulated; and 4: ERK DNM-transfected PARC-stimulated. Panel A. Western blotting of 48-hrs culture supernatants for pro-collagen type 1 (loading normalized to total protein in cell lysates from washed fibroblast monolayers in these cultures). Panel B. Western blotting for phosphor-ERK1/2 in fibroblast lysates after 15 min of stimulation with PARC. This particular antibody (from Upstate) reacts preferentially with phospho-ERK2 but not with phospho-ERK1

FIG. 8. Collagen production after activation of fibroblast cell cultures LF2 with rhPARC, p38 inhibitor SB203580, and ERK inhibitor PD98059 (A) and densitometric values of the collagen bands in the corresponding lanes (B). Collagen was metabolically labeled with 14C-proline, culture supernatants were separated in PAGE, and procollagen chains were visualized by fluorographically enhanced autoradiography.

FIG. 9. PARC activation of Sp1.

FIG. 10. Western blotting of LF1 whole cell lysates with anti phospho-ERK1/2 (top) and anti-ERK2 for loading control (bottom). Sample 1, control non-stimulated fibroblasts LF1; sample 2, fibroblasts LF1 activated for 15 min with 300 ng/ml rhPARC; sample 3, same as sample 2, plus 10 ng/ml of pertussis toxin; and sample 4, same as sample 2, plus 10 ng/ml of inactive mutant PT. Bordetella PT, but not its inactive mutant, block PARC-stimulated ERK phosphorylation, suggesting that PARC signaling is G protein-coupled.

FIG. 11. PARC receptor binding.

FIG. 12. Upstream signaling from PARC receptor.

FIG. 13. TGF-β1 protein and mRNA production, mean±SD, by primary lung fibroblast cultures with and without treatment with 300 ng/ml rhPARC. Each experiment was repeated at least three times, in triplicates, for all primary fibroblast cultures, with similar results. In spite of heterogeneous amplitude and timing of response to stimulation with PARC, individual fibroblast cultures demonstrated common tendencies in response to stimulation with PARC. Representative results are shown. Panels A and B. Levels of total TGF-β1 in lung fibroblast culture supernatants were measured by ELISA. Panel A shows the kinetics of total TGF-β1 levels in non-stimulated and PARC-treated LF1 cultures. Panel B shows PARC-dependent inhibition of increase in autocrine TGF-β1 after 24 and 48 hours of fibroblast activation with PARC. In panel B, levels of TGF-β1 in fibroblast supernatants at 0 hrs were subtracted from the levels at 24 and 48 hrs to calculate the increase in TGF-β1 production. Then, the value of the increase in the presence of PARC was divided by the value of the increase in control non-stimulated cultures and expressed as percent levels of TGF-β1 in the presence of PARC compared to the non-stimulated controls. Panels C and D. Real-time PCR for TGF-β1 mRNA and collagen αa2(I) mRNA levels in LF1 (panel C) and LF4 (panel D). Standard deviations (SD) are not shown for clarity; in all cases SD did not exceed 1.7 fold. Although the kinetics and amplitude of collagen mRNA increase differs between LF1 and LF4, there was no difference in TGF-β1 mRNA levels between PARC-activated and control cells in either case. Panels E and F. Mink lung epithelial cell proliferation assays in LF4 culture supernatants (panel E) and relative mink cell proliferation rates in the conditioned media after fibroblast stimulation with PARC versus non-stimulated controls (panel D). rhPARC did not affect proliferation of mink lung epithelial cells. In panel E, counts per minute (CPM) in thymidine incorporation assays are plotted on the vertical axis. Mink cells were cultured, from left to right, in fibroblast cell culture medium without (Ctrl) and with (Ctrl+anti-TGF-β) neutralizing anti-pan-TGF-β antibody, conditioned medium from control (LF4 Ctrl) and PARC-treated (LF4 PARC) LF4 cultures, and the latter samples with added neutralizing anti-TGF-β antibody (LF4 Ctrl+anti-TGF-β and LF4 PARC+anti-TGF-β). Proliferation rates as judged by thymidine incorporation in LF4 Ctrl and LF4 PARC samples were significantly lower (p<0.01) than in other samples, as was lower proliferation rate in LF4 PARC samples compared to LF4 Ctrl (p<0.02). In panel F, relative thymidine incorporation rates by mink cells cultured in conditioned media from PARC-stimulated fibroblast cultures were significantly lower that by mink cells cultured in conditioned media from non-stimulated control fibroblast cultures (p values shown above corresponding bars).

FIG. 14. Western blotting for collagen α1(I) of fibroblast culture supernatants after 48 hrs treatment of LF1 (panels A-D) and LF4 (panel E) with an inhibitor (lane 1), such as neutralizing pan-anti-TGF-β (panels A and B), rhLAP (panels C and D), and aprotinin (panel E), PARC (lane 2 in panels A, C, and E) or TGF-β (lane 2 in panels B and D), and PARC or TGF-β plus inhibitor (lane 3). Levels of collagen in cultures with no additives were not different from cultures with added inhibitors alone (not show). Anti-TGF-β antibody (panels A and B) and rhLAP (panels C and D) inhibited stimulating effect of TGF-β (panels B and D) but not PARC (panels A and C) on collagen production. Aprotinin also failed to inhibit PARC-stimulated upregulation of collagen levels (panel E).

FIG. 15. EMSA of nuclear lysates with Sp1 -specific probe (top) and Smad3/4 probe (bottom) after 30 minutes stimulation of LF1 with rhPARC. PARC activates transcription factor Sp1, but not Smad3/4. Samples from left to right: 1. Control fibroblasts, hot probe; 2. fibroblasts activated with PARC, hot probe; 3. Fibroblasts activated with PARC, hot+cold probes. Equal loading was controlled by measuring total protein with Bio-Rad reagent.

FIG. 16. Stimulation with PARC increases Sp1 promoter-stimulating activity in cultured primary lung fibroblasts. Luciferase activity was measured in a chemiluminescence-based assay in cell lysates from the following fibroblast cultures: mock-transfected non-stimulated (Medium), pGAM-transfected non-stimulated (pGAM Ctrl), pGAM-transfected PARC-stimulated (PGAM PARC), pGAGC6-transfected non-stimulated (pGAGC6 Ctrl), and pGAGC6-transfected PARC-stimulated (pGAGC6 PARC). Stimulation of fibroblasts with PARC leads to activation of the Sp1-sensitive promoter in pGAGC6.

FIG. 17. Western blot with anti-phosphoserine antibody (panels A,C), anti-Sp1 antibody (panel B), and anti-Smad2/3 antibody (panel D) of fibroblast lysates after Sp1 (panels A,B) and Smad3 (panels C,D) immunoprecipitation. Fibroblast cultures were activated with rhPARC for indicated times. Phosphorylation of Sp1 and Smad3 was analyzed in LF1 and LF4 on two different occasions in each culture. Increase phosphorylation of Sp1, as indicated by increase in band density after PARC stimulation was statistically significant at 30 and 90 min, and at 3 hrs (p<0.05 by one-way ANOVA of combined densitometry data from four experiments) and close to statistically significant at 24 hrs (p=0.11g).

FIG. 18. Electromobility shift assay using ³²P-labeled Sp1-specific probe. Panel A. Lanes 1-5: nuclear lysates hybridized with the Sp1 probe after 0 min, 45 min, 90 min, 3 hrs, and 24 hrs incubation of fibroblast cultures with rhPARC. Lane 6: same as lane 1, with anti-Sp1 antibody added; lane 7: same as lane 1, with 10-fold excess mutant cold probe added; lane 8: same as lane 1, with 10-fold excess cold probe added. Panel B. Density of the band indicated by the arrow in the samples 1-5, arbitrary units. These experiments were repeated at least two times with similar results in LF1, LF2, and LF4. Fold-increase of the Sp1 band density after PARC stimulation was significant within the first 3 hours of activation with PARC (p<0.05 by one-way ANOVA) and close to significant (p<0.09) after 24 hours of activation. Panel C. Nuclear lysates hybridized with the Smad3/4 probe after incubation of fibroblast cultures with rhPARC for indicated times, or in cell culture medium with no additives (Ctrl) or with TGF-β for 30 min; the sample in the lane marked “cold inhibitor” was incubated with 10-fold excess cold probe. These experiments were repeated two times in LF1 and one time in LF2 and LF4; the densities of the specific Smad3 band in PARC-stimulated samples were within 0.9-1.2 fold of the density at time 0.

FIG. 19. Western blot of LF4 culture supernatants for collagen type I. Loading was normalized to total protein in cell lysates from washed fibroblast monolayers. Panels A and B. Transient transfection with the control plasmid pcDNA3 (panel A) or mock transfection (panel B) did not affect PARC-stimulated upregulation of collagen level (compare lane 2 with lane 1). Transfection of fibroblast cultures with 1 μg of dominant negative mutant constructs for Sp1 (panel A) and Smad3 (panel B) abrogated basal production of collagen (lane 3) and the response to PARC (lane 4) in these cultures (repeated twice in LF1 and LF4 with similar results). Transfection with 0.2 μg of the dominant negative constructs did not affect basal collagen production but inhibited the response to PARC (lanes 5 and 6). Panel C. Both wild-type (wt) and Smad3-/-mouse fibroblasts produced basal collagen (lanes 1 and 3, respectively), but only wild-type fibroblasts responded to PARC stimulation by upregulating collagen production (lane 2, compare to lane 4 for PARC-stimulated Smad3-fibroblasts). Panels D and E. ALK5 inhibitor SB431542 abrogated TGF-β-stimulated (panel E) but not PARC-stimulated (panel F) collagen upregulation. These pharmacologic inhibition experiments were repeated at least twice in each of the four tested cultures, with consistent results.

FIG. 20. Western blotting for collagen type I. In each panel, cells were treated for 48 hrs with an inhibitor alone (lane 1), cytokine (lane 2), or inhibitor and cytokine combined (lane 3). Inhibitors and cytokines are indicated below each panel. Levels of collagen in cultures with no additives were not different from cultures with added inhibitors alone (not shown). Anti-TGF-β antibody inhibited effects of TGF-β (panel B) and MCP-1 (panel C) but not PARC (panel A). Recombinant human LAP inhibited stimulating effect of TGF-β (panel E) but not PARC (panel D) on collagen production. Aprotinin also failed to inhibit PARC-stimulated upregulation of collagen levels (panel F) but had no effect on collagen production in fibroblast cultures stimulated with rhTGF-β (not shown), as this cytokine is already in the active form. Experiments with neutralizing anti-TGF-β antibodies were repeated in LF1, LF3, and LF4 on at least two occasions in each of these cultures with consistent results. Neutralizing experiments with rhLAP and aprotinin were repeated at least twice in LF1 and LF4 with consistent results. Densities of the scanned bands were significantly reduced in the samples represented by lane 3 compared with lane 2 in panels B, C and E (p<0.05) but not in panels A, D, and F (p>0.05).

### DETAILED DESCRIPTION OF THE INVENTION

The present invention relates to methods of treating, preventing or preventing the progression of fibrosis comprising inhibiting the actions of pulmonary and activation-regulated chemokine (PARC) or at least one of its downstream effector molecules, such as, but not limited to, Sp1 transcription factor and protein kinase C-alpha (PKCα). As used in relation to fibrosis, the term “treatment” is used to indicate a procedure which is designed to ameliorate one or more causes, symptoms, or untoward effects of an abnormal condition in a subject. Likewise, the term “treat” is used to indicate performing a treatment. The treatment can, but need not, cure the subject, i.e., remove the cause(s), or remove entirely the symptom(s) and/or untoward effect(s) of the abnormal condition in the subject. Thus, a treatment may include treating a subject to attenuate symptoms such as, but not limited to, discomfort, pain, shortness of breath, chronic hacking cough, fatigue and weakness, loss of appetite, rapid weight loss and even death in a subject, or may include removing or decreasing the severity of the root cause of the abnormal condition in the subject. Symptoms of fibrosis, and their severity, will vary from subject to subject. A trained physician or veterinarian, however, should be able to diagnose fibrosis in a subject with proper and accurate testing, the results of which may also be considered symptoms or indicative of causes of fibrosis. Similarly, the term “prevent,” as it relates to fibrosis, is used herein to mean performing a procedure which is designed to prohibit one or more symptoms of fibrosis from detectably appearing. Of course, the term “prevent” also encompasses prohibiting entirely a fibrotic condition, or any of its associated symptoms, from detectably appearing. The phrase “preventing the progression,” as it relates to fibrosis, is used to mean a procedure designed to prohibit the detectable appearance of one or more additional symptoms of fibrosis in a patient already exhibiting one or more symptoms of a fibrotic condition, or it also means prohibiting the already-present symptoms of fibrosis from worsening in the subject. As used herein, the term “subject” is used interchangeably with the term “patient,” and is used to mean an animal, in particular a mammal, and even more particularly a non-human or human primate.

The term “fibrosis” is used to indicate an abnormal condition in a subject that is marked by excessive accumulation of collagenous connective tissue. Thus a molecule that “promotes fibrosis” is a molecule that directly or indirectly contributes to the accumulation of collagenous tissue. Examples of pathologic and excessive fibrotic accumulations include, but are not limited to, pulmonary fibrosis, benign prostate hypertrophy, fibrocystic breast disease, uterine fibroids, ovarian cysts, endometriosis, coronary infarcts, cerebral infarcts, myocardial fibrosis, musculoskeletal fibrosis, post-surgical adhesions, liver fibrosis, cirrhosis, real fibrotic disease, fibrotic vascular disease, e.g., atherosclerosis, varix, or varicose veins, scleroderma, Alzheimer's disease, diabetic retinopathy and glaucoma to name a few. In one embodiment of the present invention, the fibrosis that is treated, prevented or prevented from progressing by the methods described herein is pulmonary fibrosis. In more particular embodiments, the pulmonary fibrosis is a symptom of a condition such as, but not limited to, scleroderma, sarcoidosis, hypersensitivity pneumonitis, rheumatoid arthritis, lupus, asbestosis, and idiopathic pulmonary fibrosis.

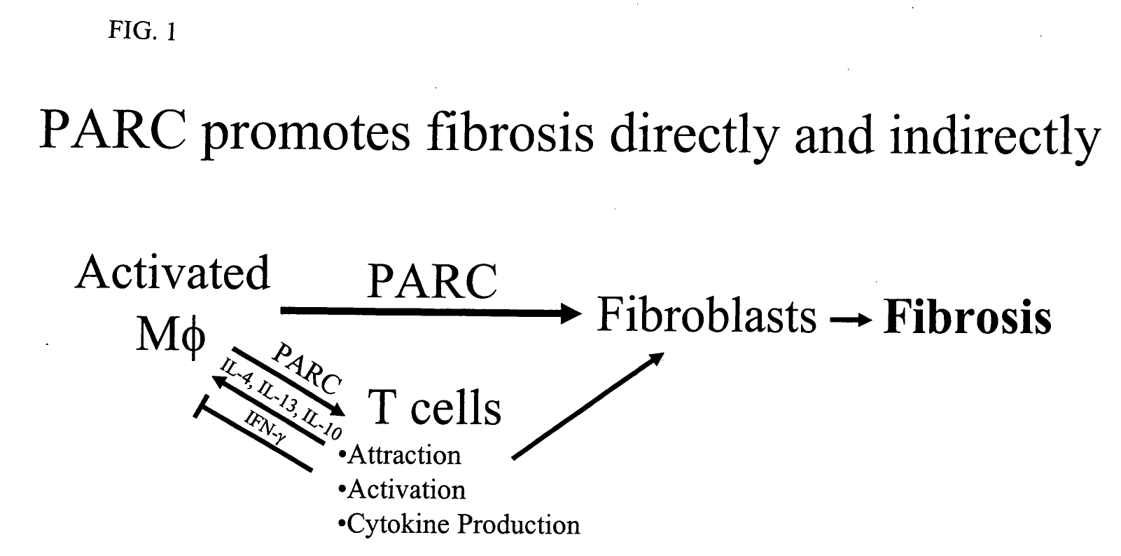

Inhibiting pulmonary and activation-regulated chemokine (PARC) is useful in treating, preventing or preventing the progression of fibrosis. PARC is a profibrotic “CC chemokine” that is chemotactic for T cells that is constitutively expressed in the lungs. PARC has a high amino acid sequence identity with macrophage inflammatory protein-1 alpha (MIP-1α), but does not bind to the MIP-1α receptors CCR5 and CCR1. Monocyte chemotactic protein-1 (MCP-1) is the only other known CC chemokine capable of increasing collagen production in fibroblasts. As shown in FIG. 1, PARC promotes fibrosis in both direct and indirect pathways. As reflected in its name, PARC is expressed at high levels in the lungs, particularly by activated lung macrophages, although other tissue macrophages and dendritic cells can secrete PARC.

In addition to its profibrotic activity, PARC attracts naive and activated CD4+ and CD8+ T cells. Indeed, fibrosis was observed in animals infected with a replication-deficient adenovirus harboring the PARC gene. Generally, it is thought that adenoviral systems of cytokine delivery do not provide high enough expression of the protein to directly stimulate collagen production in macrophages. The levels of PARC produced in adenoviral models may, however, be sufficient to attract T-cells, which may, in turn, be contributing to collagen accumulation and fibrosis observed in these adenovirus-infected animals. Accordingly, the present invention also provides methods of treating, preventing or preventing the progression of fibrosis in a subject by inhibiting the activation, growth, differentiation or movement of leukocytes, in particular lymphocytes and even more in particular T-cells.

PARC is a cytokine that is differentially regulated in classically and alternatively activated macrophages. For example, interferon-γ inhibits PARC production in activated macrophages, whereas interleukin 4 (IL-4), IL-13, and IL- 10 induce PARC production. Furthermore, the development of pulmonary fibrosis is generally associated with predominant expression of type 2 cytokines in the lungs, thus type 2 cytokines not only promote lung fibrosis by acting directly on lung fibroblasts, but also indirectly through alternative pathway to increase production of PARC.

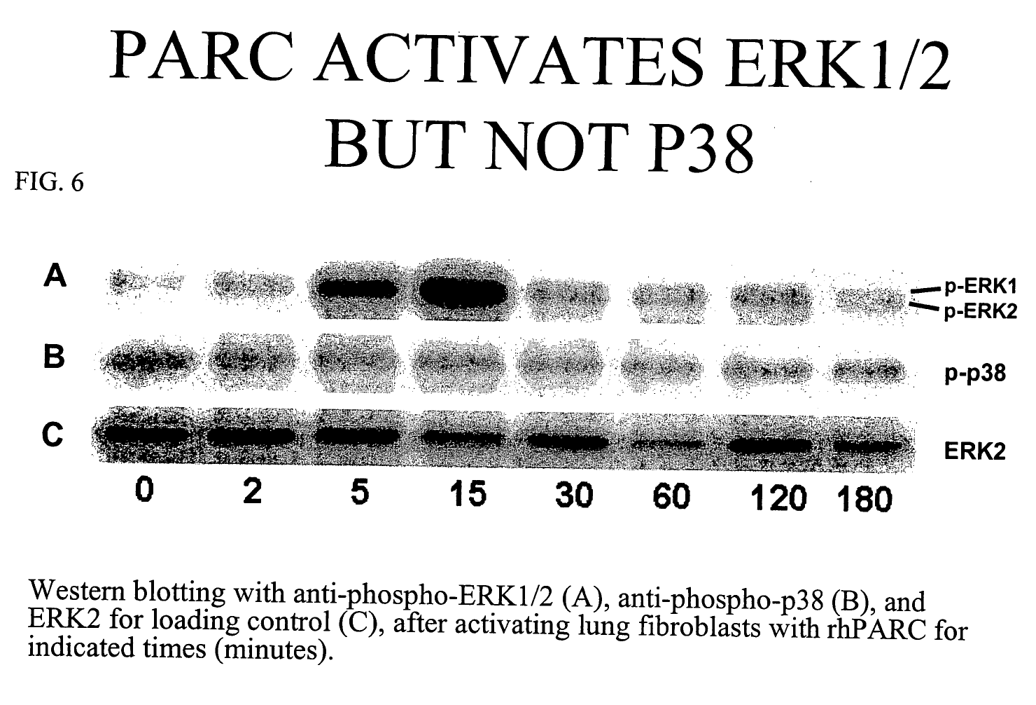

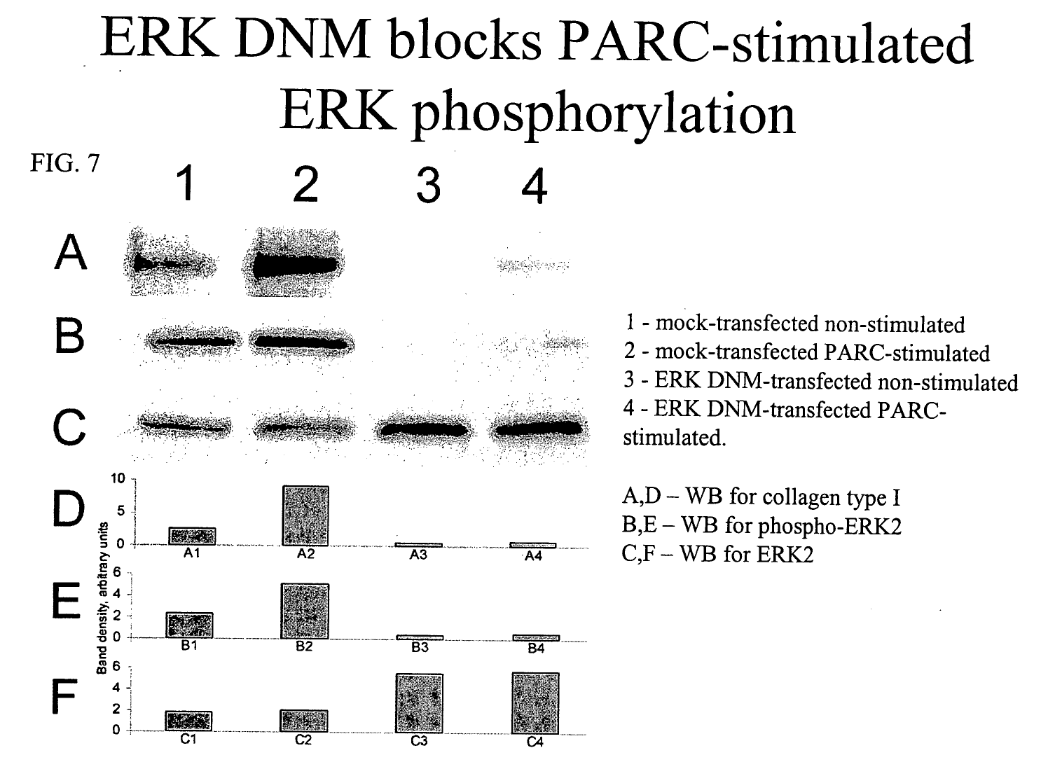

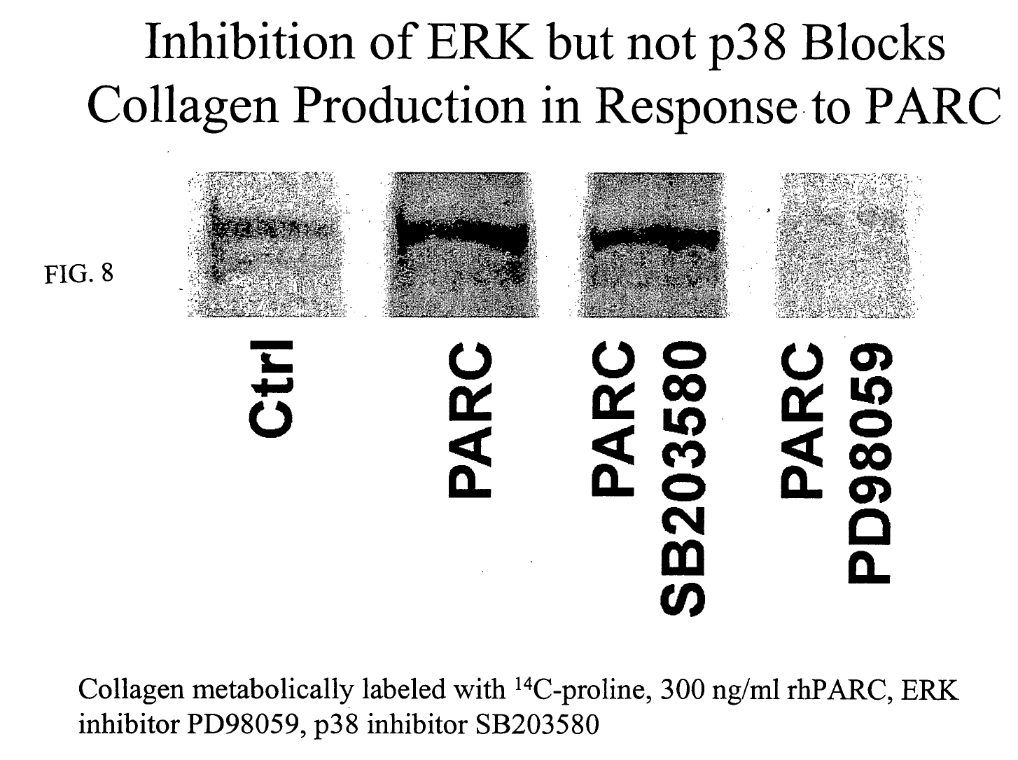

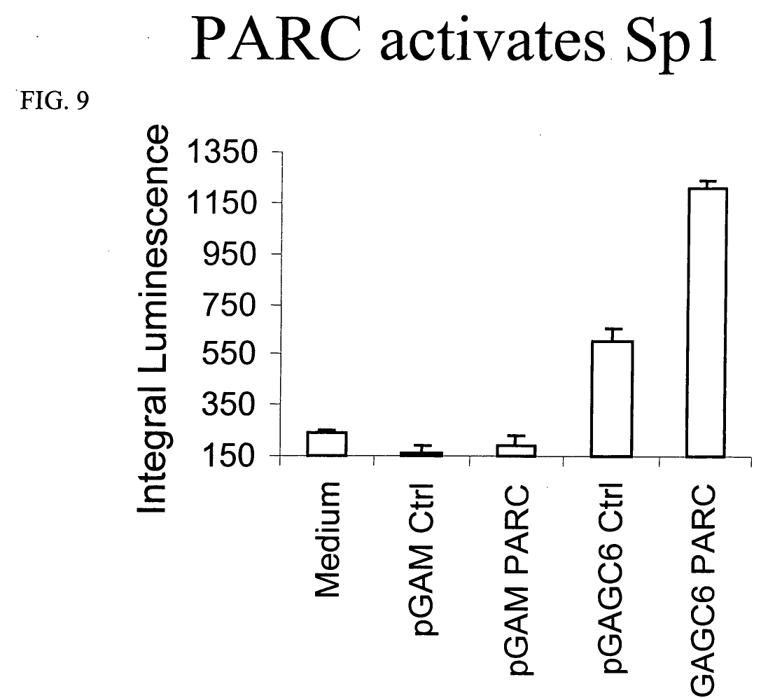

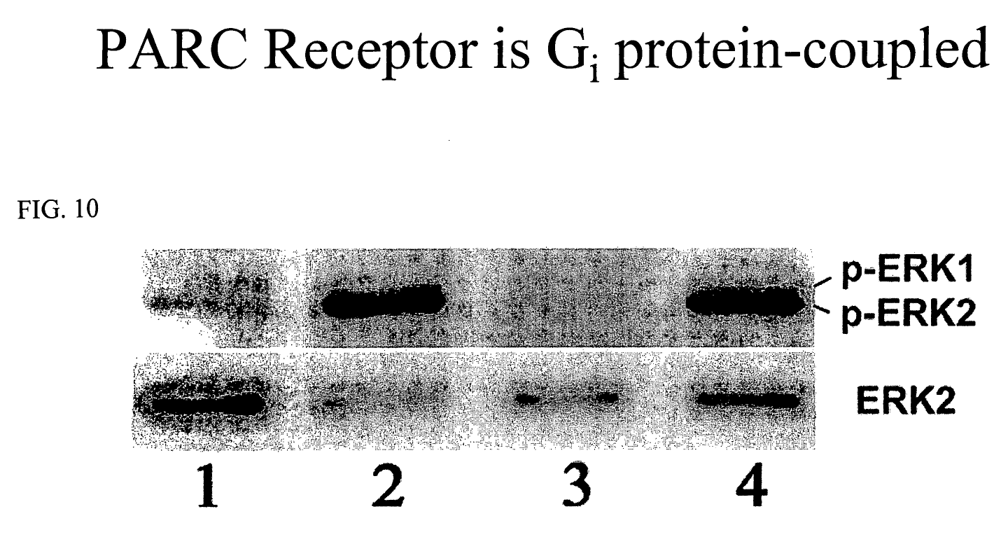

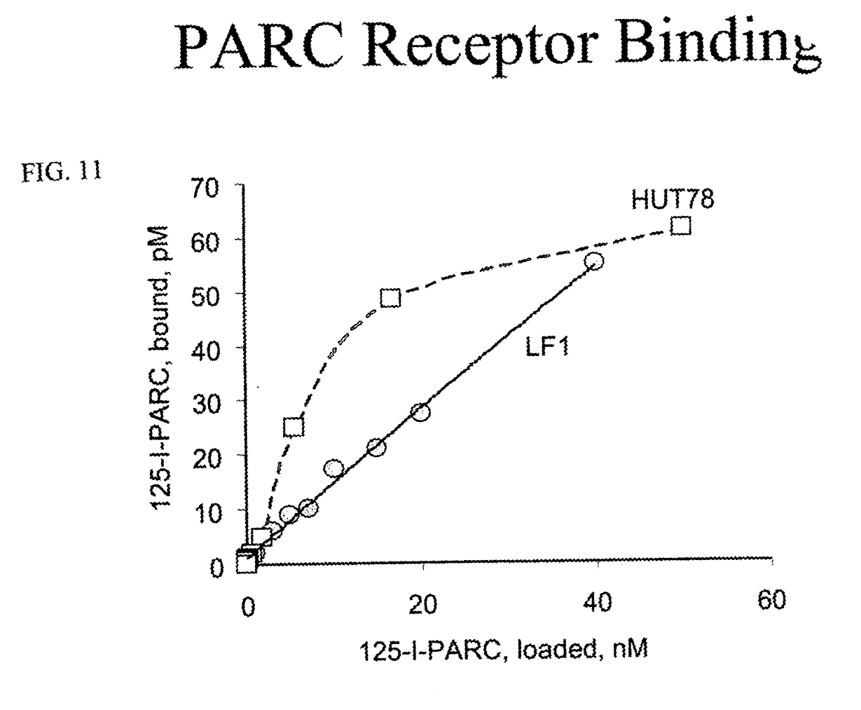

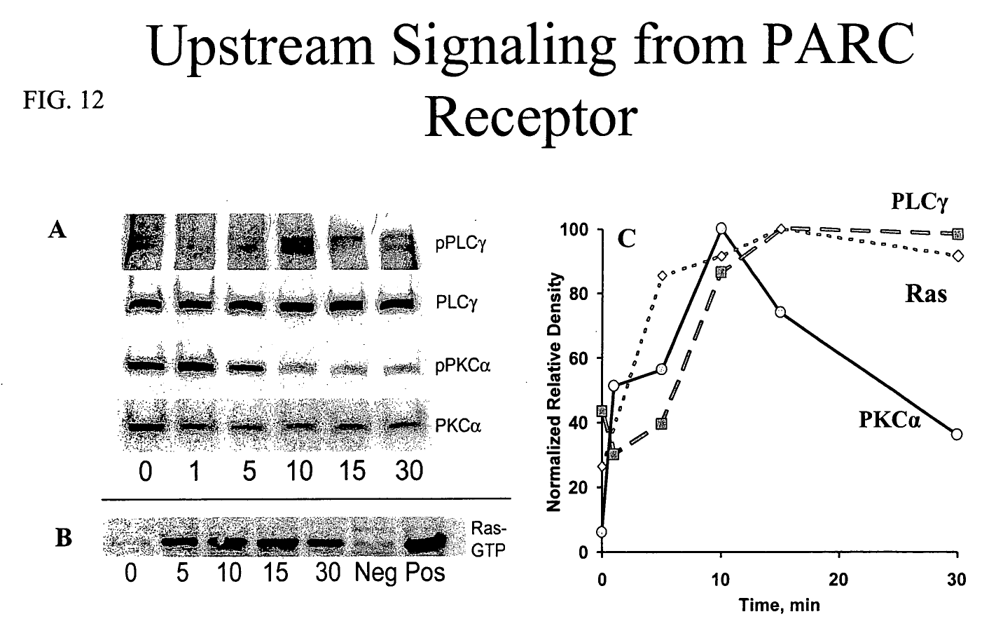

PARC increases the phosphorylation of extracellular signal-regulated kinase (ERK), a kinase involved in a variety of second messenger cell signaling cascades, in a time-dependent manner (FIG. 6). In addition, pharmacological inhibition of ERK blocked the PARC-induced stimulation of collagen production in fibroblasts (FIG. 8). Accordingly, PARC directly stimulates collagen production in lung and dermal fibroblasts by activating intracellular signaling through the ERK pathway. In addition, the PARC receptor is DRY-12, a G-coupled protein (FIG. 10) that is similar to receptors for other CC chemokines. Indeed, ERK signaling, activated via a G-coupled protein receptor, is a common signaling pathway for CC chemokines.

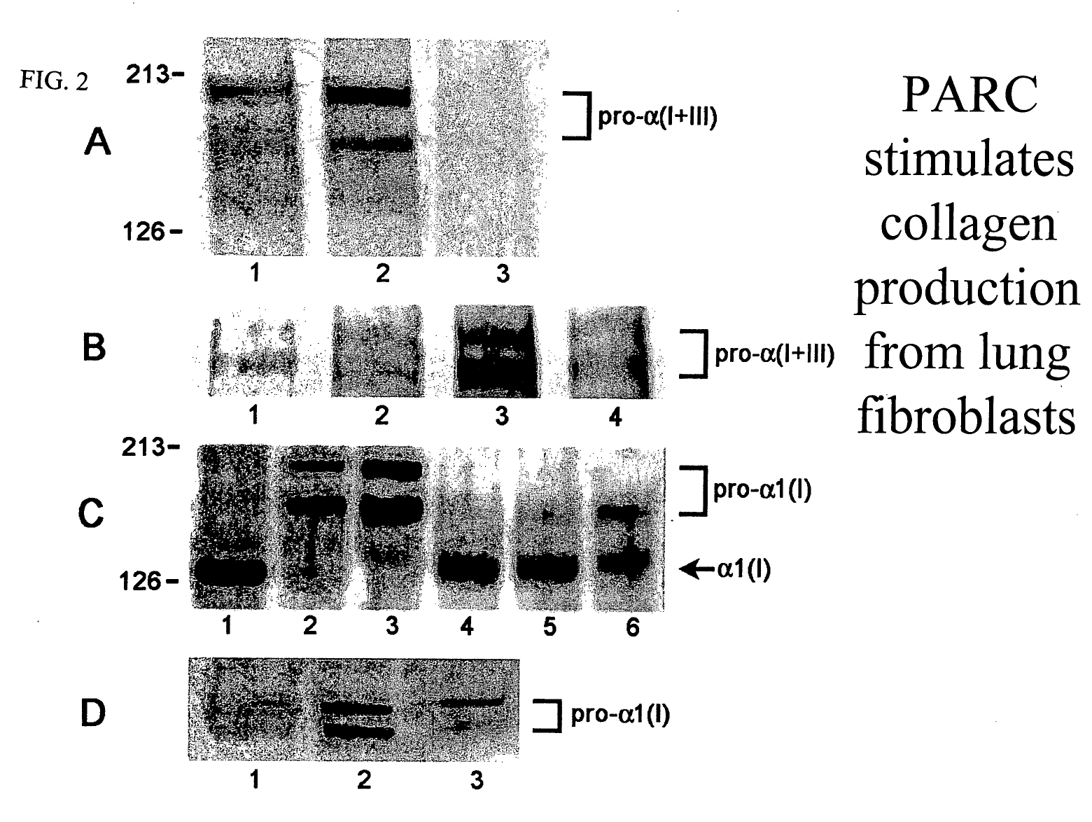

As mentioned, PARC directly stimulates type I collagen production in at least lung and dermal fibroblasts. At the same time, PARC causes only 25-50% increase in fibroblast proliferation. Both collagen protein and collagen mRNA are upregulated in fibroblasts activated with PARC (FIGS. 2-4). This increase in collagen mRNA indicates that either an increase in gene transcription or an increase in mRNA stability may be responsible for the increased collagen production in response to PARC. It is possible that PARC may also effect the intracellular pools of free proline, thus accounting for, at least in part, PARC's stimulation of collagen production in fibroblasts.

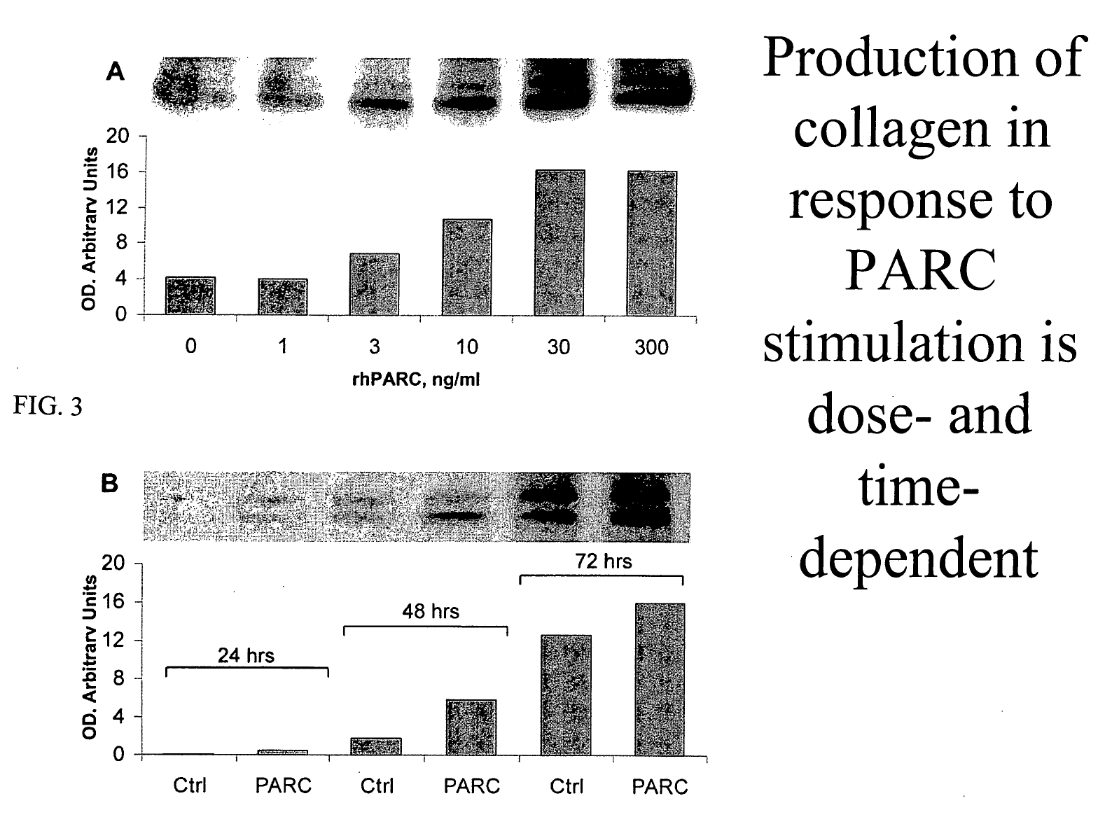

The concentrations of PARC required to achieve a significant effect on collagen production are compatible with those reported for MCP-1, with PARC causing detectable stimulation of collagen production in concentrations below about 300 ng/ml (FIG. 3), and MCP-1 stimulating detectable collagen production in concentrations of about 100-400 ng/ml. Considering that activated alveolar macrophages are an abundant source of PARC, and that lung macrophages are actively involved in lung inflammation involved in pulmonary fibrosis, PARC is likely present during lung inflammation in amounts sufficient to stimulate fibrosis. Indeed, studies have shown that PARC has been found in elevated concentrations in BAL fluids taken from patients with scleroderma lung disease.

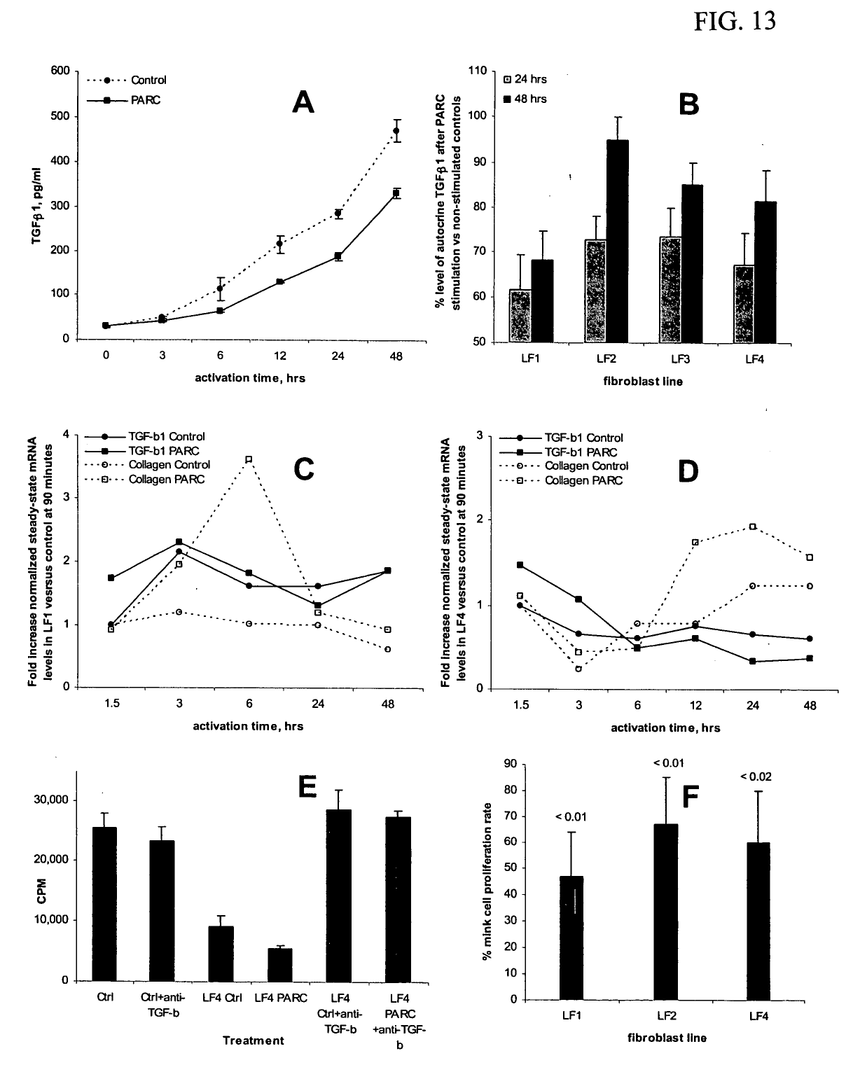

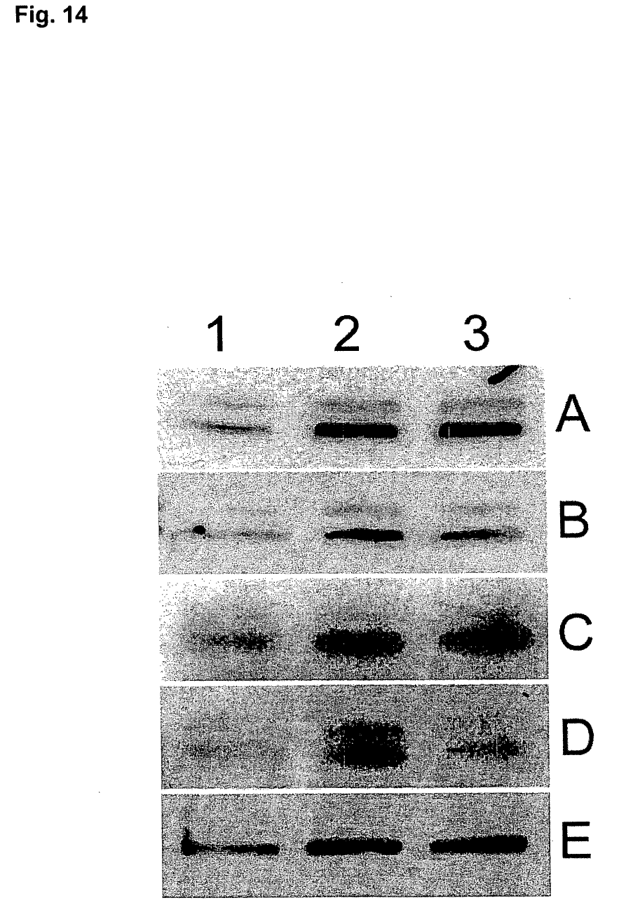

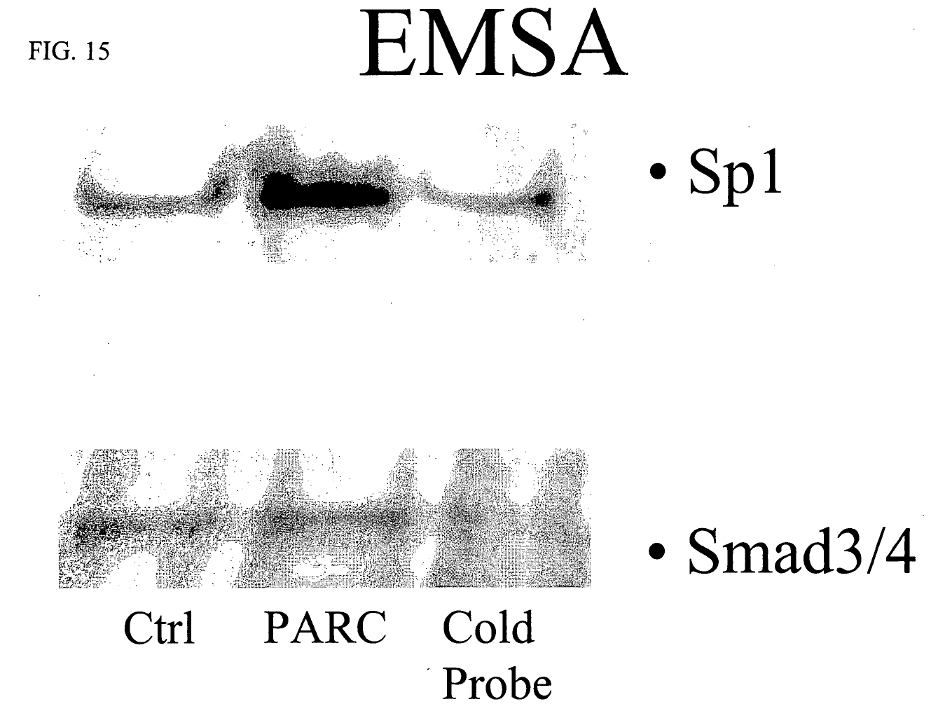

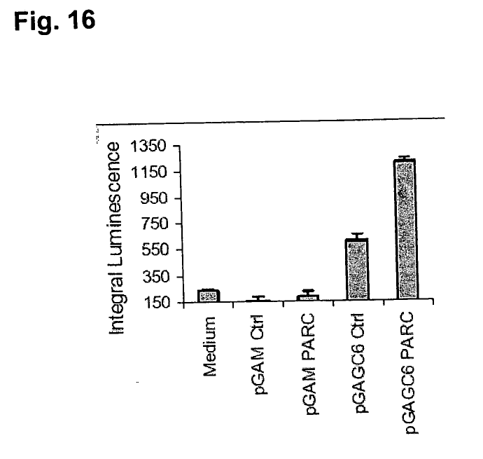

It was generally believed that TGF-β must have a role in pulmonary fibrosis; however, no increase in TGF-β protein was found in cell culture supernatants or in whole cell lysates of fibroblast lines that were stimulated with recombinant human PARC (rhPARC). Indeed, levels of total TGF-β1 protein were decreased in the PARC-treated cultures (FIG. 13A,B), and there was no difference in steady-state levels of TGF-β1 mRNA between PARC-treated and control cultures (FIG. 13C,D). The lower levels of TGF-β1 protein in the PARC-treated cultures were unexpected. To determine if PARC accelerates the activation of TGF-β, thus accounting for the increase in collagen production in spite of the apparent decrease in levels of TGF-β, a functional assay using an anti-TGF-β antibody was employed. Specifically, active TGF-β inhibits the proliferation of mink lung epithelial cells (FIG. 13E, F), and culture supernatants of LF1, LF2, and LF4 cells, which secrete activated TGF-β, are able to inhibit the proliferation of these cells. Supernatant from PARC-treated cells showed almost no difference in its inhibitory effects of the proliferation of epithelial cells, indicating that PARC has practically no effect on the levels of active TGF-β (FIG. 13E,F) secreted from lung fibroblast cells. These observations demonstrate that PARC does not alter the levels of active TGF-β in lung fibroblasts to any appreciable extent, which indicates that PARC's stimulation of collagen in lung fibroblasts is independent of activated TGF-β. In addition, FIG. 14 demonstrates that PARC does not appear to consume or degrade levels of TGF-β. Thus, although surprising, TGF-β does appear to be involved in any of PARC's effects on collagen production in lung fibroblasts.

Accordingly, certain aspects of the present invention relate to various methods of treating, preventing or preventing the progression of fibrosis in a patient comprising inhibiting the activity of an activator molecule, other than TGF-β, that promotes fibrosis or symptoms thereof. In particular, certain aspects of the methods of the present invention relate to inhibiting the activity of PARC and/or its downstream effector molecules, where the inhibition is substantially limited to PARC and/or its downstream effector molecules. In another aspect, the invention provides methods of treating, preventing or preventing the progression of fibrosis in a patient comprising inhibiting the activity of PARC in combination with inhibiting the activity of TGF-β. In one particular embodiment, the methods of inhibiting both PARC and TGF-β may comprise administering a single active compound that inhibits both PARC and TGF-β. In another particular embodiment, the methods of inhibiting both TGF-β and PARC comprise coadministering more than one active compound, which may or may not be in admixture together. As used herein, the term “coadminister” is used to mean that each of at least two compounds are administered during a time frame wherein the respective periods of biological activity or effects overlap. Thus the term includes sequential as well as coextensive administration of the compounds of the present invention.

Alternatively, certain aspects of the present invention relate to methods of treating, preventing or preventing the progression of fibrosis in a patient, where the methods are substantially ineffective against the activity of TGF-β. In one particular embodiment, the present invention relates to methods of treating, preventing or preventing the progression of fibrosis in a patient, without detectably affecting the local or systemic activity of TGF-β. Local activity of TGF-β includes, but is not limited to, such locations as the skin or respiratory system.

As used herein, the term “inhibit” is used to mean that the treatment confers detectable decrease in activity or effects of a molecule, compared to that of the untreated molecule. The inhibition may be complete inhibition, i.e., no detectable activity is observed after treatment, or it may simply be partially reduced. For example, if the activity of a particular includes phosphorylation of target proteins or molecules, inhibiting would comprise detectably decreasing the phosphorylation of target molecules, which could directly or indirectly be assayed. Similarly, if the activity of a particular molecule includes DNA binding, inhibiting this activity would comprise modulating the DNA-binding molecule such that there is a detectable decrease in the binding of the molecule to nucleic acids, which could be directly or indirectly assayed.

The scope of the invention is not limited to particular methods of inhibiting the activity of PARC. The activity of PARC may be inhibited by “gene silencing methods” which are generally regarded as methods that prevent or decrease the rate of transcription or translation of a protein within a cell. Such gene silencing methods include, but are not limited to antisense technology, RNA inhibition technology (RNAi) and inactivation or degradation of transcription factors required for PARC transcription etc.

Generally, RNAi technology is limited to tissue-specific or organ-specific areas of the subject using tissue-specific gene promoters or transcription factors. Promoters are nucleic acid that are generally located in the 5′-region of a gene, proximal to the start codon or nucleic acid which encodes untranslated RNA. The transcription of an adjacent nucleic acid segment is initiated at the promoter region. A repressible promoter's rate of transcription decreases in response to a repressing agent. An inducible promoter's rate of transcription increases in response to an inducing agent. A constitutive promoter's rate of transcription is not specifically regulated, though it can vary under the influence of general metabolic conditions. Any suitable promoter may be used to control the production of RNA from the nucleic acid molecules of the invention. Promoters may be those recognized by any polymerase enzyme; for example, promoters may be promoters for RNA polymerase II or RNA polymerase III. Examples of lung-specific promoters for proteins whose expression is generally restricted to the respiratory system include, but are not limited to, such proteins as pulmonary lung Surfactant Proteins A, B, C and D (“SPA, SPB, SPC and SPD”) and Clara cell secretory protein (“CCSP”). Other suitable promoters are known in the art and are within the scope of the present invention. Recombinant DNA methods, such as those that might be used to prepare constructs of a tissue-specific promoter operably linked to a coding region coding for messenger RNA are well known in the art.

One example of a construct designed to produce RNAi is a construct where a DNA segment is inserted into a vector such that RNA corresponding to both strands are produced as two separate transcripts. Another example of a construct designed to produce RNAi is a construct where two copies of a DNA segment are inserted into a vector such that RNA corresponding to both strands are again produced. Yet another example of a construct designed to produce RNAi is a construct where two copies of a DNA segment are inserted into a vector such that RNA corresponding to both strands are produced as a single transcript. Expression of one of these DNA segments results in the production of sense RNA while expression of the other results in the production of an anti-sense RNA.

Nucleic acid segments designed to produce RNAi need not correspond to the full-length gene or open reading frame. For example, when the nucleic acid segment corresponds to an open reading frame (ORF), the segment may only correspond to part of the ORF (e.g., about 50 nucleotides or even fewer at the 5′ or 3′ end of the ORF).

The invention also relates to compounds and methods for gene silencing involving ribozymes. In particular, the invention provides antisense RNA/ribozymes fusions which comprise (1) antisense RNA corresponding to a target gene and (2) one or more ribozymes which cleave RNA (e.g., hammerhead ribozyme, hairpin ribozyme, delta ribozyme, Tetrahymena L-21 ribozyme, etc.). Further, provided by the invention are vectors which express these fusions, methods for producing these vectors, and methods for using these vectors to suppress protein expression.

Methods of inhibiting the activity of PARC also include, but are not limited to, decreasing the stability of mRNA coding for the PARC protein. Additional methods of inhibiting the activity of PARC include, but are not limited to, using a PARC antagonist, such as a pharmacological inhibitor of PARC, to decrease the effects PARC binding to its receptor.

Still more methods of inhibiting the activity of PARC include the use of antibodies or functional fragments thereof to either bind the PARC receptor or bind the PARC protein, which may prevent subsequent downstream signaling that normally follows the binding of PARC to its receptor. As used herein, the term “antibody” includes at least monoclonal antibodies and polyclonal antibodies; and “functional fragments” of an antibody is used mean a portion of an antibody that can bind, to some extent, at least the antigen of the fully in-tact antibody. “Functional fragments” thus includes molecules that bind more than one antigen, such as, but not limited to a tetramer of single chain fragment of variable region (scFV). Antibodies are prepared by well-known methods in the art, such as immunizing suitable mammalian hosts in appropriate immunization protocols using peptides, polypeptides or proteins of the invention if they are of sufficient length, or, if desired or required to enhance immunogenicity, they can be conjugated to suitable carriers (see, e.g., Harlow, E., and Lane, D., Antibodies: A Laboratory Manual, Cold Spring Harbor, N.Y.: Cold Spring Harbor Laboratory Press (1988); Kaufman, P. B., et al., In: Handbook of Molecular and Cellular Methods in Biology and Medicine, Boca Raton, Fla.: CRC Press, pp. 468-469 (1995)). Methods for preparing immunogenic conjugates with carriers such as bovine serum albumin (BSA), keyhole limpet hemocyanin (KLH), or other carrier proteins are well known in the art. In some circumstances, direct conjugation using, for example, carbodiimide reagents may be effective; in other instances linking reagents such as those supplied by Pierce Chemical Co., Rockford, Ill., may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

Anti-peptide antibodies can be generated using synthetic peptides. Synthetic peptides can be as small as 2-3 amino acids in length, and are suitably at least 3, 5, 10, or 15 or more amino acid residues long. Such peptides can be determined using programs such as DNAStar. The peptides can be coupled to KLH using standard methods and can be immunized into animals such as rabbits. Polyclonal anti-PARC, anti-PKCα, anti-Sp1, or other anti-effector molecule peptide antibodies can then be purified, for example using Actigel beads containing the covalently bound peptide.

While the polyclonal antisera produced in this way may be satisfactory for some applications, for pharmaceutical compositions, use of monoclonal preparations is also suitable. Immortalized cell lines which secrete the desired monoclonal antibodies may be prepared using the standard method of Kohler and Milstein or modifications which effect immortalization of lymphocytes or spleen cells, as is generally known (Kohler et al., Nature 256:495 (1975); Kohler et al., Eur. J. Immunol. 6:511 (1976); Kohler et al., Eur. J. Immunol. 6:292 (1976); Hammerling et al., In: Monoclonal Antibodies and T-Cell Hybridomas, Elsevier, New York, pp. 563-681 (1981)). The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies can then be recovered from the culture supernatant. Antibodies or functional fragments thereof can be used as antagonists of activity against PARC, PKCα, Sp1, or other PARC effector molecules. Use of functional fragments, such as the Fab, Fab′ or F(ab′)2 fragments are often suitable, especially in a therapeutic context, as these fragments are generally less immunogenic than the whole immunoglobulin.

The antibodies or fragments, such as scFV fragments, may also be produced by recombinant means. Regions that bind specifically to the desired regions of PARC or the PARC receptor or its downstream effector molecules can also be produced in the context of chimeras with multiple species origin. Antibody reagents so created are contemplated for use diagnostically or as stimulants or inhibitors of the activity of PARC or an effector molecule such as, but not limited to PKCA and Sp1.

PARC directly stimulates collagen production in fibroblasts by at least two distinct downstream effector molecules: PKCα and Sp1. As used here, the phrase “effector molecule” is used to mean a molecule that is capable of generating a signal or subsequent message (second messenger) or capable of exerting a detectable intracellular or intercellular effect on the metabolism, gene expression or proliferation of a cell or group of cells, such as but not limited to tissue or an organ. For the purposes of the present invention, the effector molecule is responsive to an “activator molecule.” Indeed, the detectable effects of effector molecules are correlative, either directly or inversely, with the presence or absence of the “activator molecules.” As used herein, an “activator molecule” is a molecule or compound that directly or indirectly initiates or inhibits the activity of at least one effector molecule. For example, an activator molecule may initiate a cascade of intracellular or intercellular events and or signals that ultimately leads to the activation or inactivation of an effector molecule, which, in turn, will affect the metabolism, gene expression or proliferation of a cell or group of cells. Accordingly, it is possible that a specific molecule may be considered to be an activator molecule as well as an effector molecule, relative to its position in a signaling cascade event. For example, the PARC receptor and PKCα would be effector molecules, relative to PARC; however, PKCα may also be considered an activator molecule relative to a transcription factor, such as, but not limited to Sp1 and Smad3. In turn Sp1, would also be an effector molecule relative to PARC.

To that end, the present invention also provides method of treating fibrosis in a patient comprising inhibiting at least one effector molecule of PARC, including, but not limited to, the Sp1 transcription factor or PKCα. Indeed, the inventors have shown that PARC stimulates collagen production in fibroblasts by activating PKCα and activating Sp1. PKCα is an isoform of the protein kinase C family serine/threonine kinases. The PKC family of isozymes generally comprises a C-terminus catalytic region, which contains the active site, and a regulatory region that contains several highly conserved domains that are responsible for cell membrane association and activation. PKC isozymes are identified and classified in the art according to structural and functional differences occurring within the conserved domains. Some isozymes, e.g., PKCα, PKCβI, PKCβII and PKCγ, possess a calcium ion binding domain and are thus dependent upon calcium for their activation. Other isozymes, e.g., PKCδ, PKCε, PKCη, PKCθ and PKCμ lack a calcium binding domain. In specific embodiments of the present invention, methods are presented to treat, prevent or prevent the progression of fibrosis in a patient, comprising inhibiting the activity of PKCα. In one particular embodiment, the methods are directed towards treating or preventing fibrosis comprising inhibiting the activity of PKCα in the respiratory system. In another particular embodiment, the methods are directed to treating, preventing or preventing the progression of fibrosis comprising inhibiting the activity of PKCα, where the inhibition is substantially limited to the alpha isozyme of the PKC family of isozymes. In an even more particular embodiment, the methods are directed to treating, preventing or preventing the progression of fibrosis comprising inhibiting the activity of PKCα, where the inhibition is substantially limited to the respiratory system and limited to the alpha isozyme of the PKC family of isozymes.

The scope of the invention is not limited to particular methods of inhibiting PKCα. The activity of PKCα may be inhibited by gene silencing methods such as, but not limited to, antisense technology, RNA inhibition technology (RNAi) and inactivation or degradation of transcription factors required for PKCα transcription etc, all of which are described elsewhere herein.

Methods of inhibiting the activity of PKCα also include, but are not limited to, decreasing the stability of mRNA coding for PKCα protein. Additional methods of inhibiting the activity of PKCα include, but are not limited to, using a PKCα antagonist, such as a pharmacological inhibitor of PKCα, to decrease the activity of the PKCα protein. Such PKCα antagonists include but are not limited to, phorbol esters, diacylglycerol kinase zeta (DKGζ) and pseudosubstrate peptides. As used herein, “pseudosubstrate peptide” is used as it is in the art; namely, it is a molecule, e.g., a peptide, that binds to a domain of the target molecule, but lacks additional structure or function to effectuate the normal activity of the target molecule. For example, a pseudosubstrate to PKCα would bind PKCα in its catalytic domain, but lack the requisite amino acids to be phosphorylated. The pseudosubstrates may or may not be modified, such as, but not limited to, glycosylation such as, for example, N-myristoylation.

Still more methods of inhibiting the activity of PKCα include the use of antibodies or functional fragments thereof that either bind PKCα or its intracellular target molecules, which may prevent subsequent downstream signaling. Methods of antibody preparation and functional fragments thereof are described elsewhere herein.

Still other methods of inhibiting the activity of PKCα include, but are not limited to, creating conditions or administering a compound that degrades the PKCα protein.

Sp1 is a transcription factor belonging to the Sp/XKLF family of transcription factors, which is generally divided into 2 major subgroups: the Sp proteins and the KLF proteins. Members of the broad family of transcription factors generally possess three conserved Cys2His2 zinc fingers that form the DNA binding sites for these transcription factors. While each member of the family is unique, it is believed that DNA binds to the Sp1 transcription factor via the KHA amino acids in the first zinc finger, RER in the second and RHK in the third finger. In specific embodiments of the present invention, methods are presented to treat, prevent or prevent the progression of fibrosis in a patient, comprising inhibiting the activity of Sp1 . In one particular embodiment, the methods are directed towards treating, preventing or preventing the progression of fibrosis comprising inhibiting the activity of Sp1 in the respiratory system. In another particular embodiment, the methods are directed to treating, preventing or preventing the progression of fibrosis comprising inhibiting the activity of Sp1, where the inhibition is substantially limited to the Sp1 transcription factor. In an even more particular embodiment, the methods are directed to treating, preventing or preventing the progression of fibrosis comprising inhibiting the activity of Sp1, where the inhibition is substantially limited to the respiratory system and to Sp1.

The scope of the invention is not limited to particular methods of inhibiting Sp1 . The activity of Sp1 may be inhibited by gene silencing methods such as, but not limited to, as antisense technology, RNA inhibition technology (RNAi) and inactivation or degradation of transcription factors required for Sp1 transcription etc, all of which are described elsewhere herein.

Methods of inhibiting the activity of Sp1 also include, but are not limited to, decreasing the stability of mRNA coding for Sp1 protein. Additional methods of inhibiting the activity of Sp1 include, but are not limited to, using a Sp1 antagonist to decrease the activity of the Sp1 protein. Such Sp1 antagonists include, but are not limited to, pseudosubstrates that bind, for example, to the DNA-binding domain(s) of Sp1, but do not code for a full-length polypeptide or protein. Other examples of pseudosubstrates to Sp1 include mimics of transcription co-factors that synergistically interact with Sp1, such as Smad3, but lack the structure or function necessary to effectuate DNA transcription. Other antagonists to Sp1 activity include, but are not limited to, other transcription factors that may bind Sp1 and repress the activity of Sp1, such as, but not limited to, pro-myelocytic leukemia protein (PML), which interacts with the C-terminus zinc finger to prevent Sp1 binding to DNA. Still other methods of inhibiting the activity of Sp1 include, but are not limited to methods that affect the glycosylation or phosphorylation state of the Sp1 protein. For example, casein kinase II phosphorylates threonine residues in the Sp1 zinc finger domains, and results in a decreased affinity of the DNA binding domain towards DNA.

Additional methods of inhibiting the activity of Sp1 include the use of antibodies or functional fragments thereof that either bind Sp1 or its intracellular target molecules, e.g., co-transcription factors or nucleotide sequences, which may prevent subsequent downstream signaling, such as DNA transcription. Methods of antibody preparation and functional fragments thereof are described elsewhere herein.

Still more methods of inhibiting the activity of Sp1 include, but are not limited to, creating conditions or administering a compound that degrades the Sp1 protein. For example, stimulation of adenylate cyclase along with glucose deprivation renders Sp1 susceptible to protease degradation.

Other downstream effector molecules of PARC include, but are not limited to, the PARC receptor, ERK1, ERK2, Smad3, Smad4, phospholipase C-gamma (PLCγ) and Ras. Thus, one aspect of the present invention also relates to methods of treating, preventing or preventing the progression of fibrosis comprising inhibiting the activity of at least one downstream effector molecule of PARC selected from the group consisting of ERK1, ERK2, Smad3, PLCγ and Ras. Similar to the methods of inhibiting the activity of PARC, PKCα and Sp1 described herein, the methods of inhibiting the activity of additional effector molecules include, but are not limited to, pharmacological means, gene silencing methods, antibodies and functional fragments thereof, pseudosubstrates and the creation of conditions that promote specific protein degradation or inactivation.

Certain embodiments of the present invention relate to administering a pharmaceutically effective amount of a medicament substance that is capable of treating, preventing or preventing the progression of fibrosis. A medicament useful for the methods of treating, preventing or preventing the progression of fibrosis may be prepared by standard pharmaceutical techniques known in the art, depending upon the mode of administration and the particular disease to be treated. The medicament will usually be supplied as part of a sterile, pharmaceutical composition which will normally include a pharmaceutically acceptable carrier. This pharmaceutical composition may be in any suitable form, (depending upon the desired method of administering it to a subject). It may be provided in unit dosage form, will generally be provided in a sealed container and may be provided as part of a kit, which may include instructions for use and/or a plurality of unit dosage forms.

The pharmaceutical composition may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by admixing the active ingredient with the carrier(s) or excipient(s) under sterile conditions.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions oils (e.g. vegetable oils) may be used to provide oil-in-water or water in oil suspensions. In certain situations, delayed release preparations may be advantageous and compositions which can deliver, for example, AET or a derivative thereof in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3 (6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurized aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets. The pharmaceutical compositions may contain preserving agents, solubilising agents, stabilising agents, wetting agents, emulsifiers, sweeteners, colourants, odourants, salts (substances of the present invention may themselves be provided in the form of a pharmaceutically acceptable salt), buffers, coating agents or antioxidants. They may also contain therapeutically active agents in addition to the substance of the present invention.

Dosages of the substance of the present invention can vary between wide limits, depending upon the disease or disorder to be treated, the age and condition of the individual to be treated, etc. and a physician will ultimately determine appropriate dosages to be used.

Because of the role that PARC and its effector molecules play in fibrosis, it is desirable to identify substances that inhibit their activity. Accordingly, the present invention also relates to methods of screening and/or identifying compounds useful for treating, preventing or preventing the progression of fibrosis. Specifically, the methods of identifying such inhibitory substances comprise (a) providing a test substance to a cell, wherein the cell possesses PARC activity, (b) measuring the amount of PARC activity in the test cell; and (c) comparing the amount of PARC activity in a control cell to which has not been provided the test substance with the amount of PARC activity in the test cell, wherein a decreased amount PARC activity in the test cell, compared to the amount of PARC activity in the control cell, indicates that the test substance inhibits PARC activity. As used herein, the terms “substance” “agent” and “compound” may be used interchangeably. The types of substances that may be assayed for their ability to inhibit PARC and/or its effector molecules include, but are not limited to, carbohydrates such as monosaccharides, disaccharides, oligosaccharides and polysaccharides, proteins, peptides and amino acids, including, but not limited to, oligopeptides, polypeptides and mature proteins, nucleic acids, oligonucleotides, polynucleotides, lipids, fatty acids, lipoproteins, proteoglycans, glycoproteins, organic compounds, inorganic compounds, ions, and synthetic and natural polymers.

As used herein, “PARC activity” is assessed by direct or indirect means. For example, PARC activity can be directly assessed by measuring or quantifying levels of PARC protein that binds to a receptor, or is produced by a cell; and PARC activity can be indirectly assessed by measuring or quantifying a detectable effect that PARC protein has on a cell. Detectable effects that PARC has on a cell encompass RNA transcription, protein expression or secretion, such as, but not limited to, collagen expression, or the generation of second messenger or intracellular signals. Thus the invention also provides methods of identifying substances useful for inhibiting collagen production or accumulation.

As stated previously, PARC is a chemokine, thus PARC activity will also include activities normally associated with chemokines, such as activity associated with (1) mediating natural immunity; (2) regulating lymphocyte activation, growth and differentiation; (3) regulating immune-related inflammation; (4) stimulating leukocyte growth and differentiation and (5) stimulating leukocyte movement. Accordingly, the invention provides for methods of identifying substances which modulate natural immunity, modulate the activation, growth and/or differentiation of lymphocytes, modulate immune related inflammation, modulate the activation, growth and/or differentiation of leukocytes and modulate the stimulation of leukocyte movement. In one particular embodiment, the present invention relates to methods of identifying substances that modulate the activation, growth, differentiation or movement of lymphocytes, in particular T-cells.

PARC activity can be assessed by other means that include, but are not limited to, phosphorylation of second messenger molecules, such as phospholipase C, adenylate cyclase and protein kinase C among others, generation of other second messenger signals such as Ca⁺² release, calmodulin binding, inositol triphosphatase activity, GTPase activating protein (GAP) activity to name a few. Other indirect measures of PARC activity include activation of transcription factors, such as, but not limited to Sp1, Smad3 and Smad4 to name a few, and levels of mRNA of specific transcripts. Other detectable effects of PARC activity encompass assessing the ability of a substance to bind to the PARC receptor, and can be assayed by traditional procedures such as, but not limited to, competitive binding assays.

The scope of the invention is not limited to means of measuring PARC activity for the purposes of comparing test substances. Thus, in one embodiment, the present invention provides methods of identifying inhibitory substances useful for treating, preventing or preventing the progression of fibrosis, with the methods comprising (a) providing a test substance to a cell, wherein the cell possesses PARC activity as measured by a means of measuring the PARC activity, and (b) comparing the amount of PARC activity, as assessed by the measuring means, in a control cell to which has not been provided the test substance, with the amount of PARC activity in the test cell, wherein a decrease in the amount PARC activity in the test cell, compared to the amount of PARC activity in the control cell, indicates that the test substance inhibits PARC activity. The measuring means may be directly correlative or inversely correlative, so long as the measuring means provides the technician with a means of assessing the levels of PARC activity in test cells that can be compared to levels of PARC activity in control cells.

As used herein the term “cell” is used to indicate one or more cells, and can be used interchangeably with the term “cells”, “cell culture” and “cell line.” In addition, the cells used in the screening methods can be isolated cells in an in vitro cell culture, or the cells may be in situ, as part of an organ or tissue; or the cells may be in vivo as part of an organ or tissue in a live subject, such as, but not limited to a mouse, rat, dog or non-human primate. The cells used in the screening methods may also be manipulated, modified, fixed or even lysed at any time during the screening process, for example, subsequent to application of the test substance, but prior to measuring the activity to be assessed. Provided that assayed activity can be measured (e.g., PARC activity, PKCα activity and Sp1 activity), the cells can thus be prokaryotic or eukaryotic, including but not limited to bacterial cells, insect cells, mammalian cells, and even plant cells. A “test cell” is a cell to which a test substance has been applied; and “control cell” is a cell to which the same test substance has not been applied. The control cell may or may not be a genetically, phenotypically or metabolically normal cell, but the control cell should be the same cell type as the test cell.

As used herein, the measurement of the activity to be assayed, for example, PARC activity, PKCα activity, Sp1 activity, etc., can be a relative or absolute measurement. Of course, the measurement of activity may be equal to zero, indicating the absence of activity. The measurement of activity may be a simple value, without any additional measurements or manipulations. Alternatively, the measurement of activity may be expressed as a difference, percentage or ratio of the measured activity to another value, but not limited to, a normal, baseline or standard measurement. The difference may be negative, indicating a decrease in the amount of measured activity. The measurement of activity may also be expressed as a difference or ratio of the activity to itself, measured at a different point in time. The measurement of activity may be determined directly, or the value of the measured activity may be used in an algorithm, with the algorithm designed to correlate the measurement to the level of activity in the cell.

In addition, the scope of the invention is not limited to the source, location or identity of the cells used in the screening methods of the present invention. Indeed, the cells may be isolated from bronchoalveolar lavage (BAL) procedures, or the cells may be isolated from patient biopsies, or they may be isolated from whole organ or organ systems such as, but not limited to, the lungs, from a subject. In one embodiment of the present invention, the cells used in the screening methods are isolated from a transgenic animal. In a more specific embodiment, the transgenic animal from which the cells are isolated has at least one copy of PARC incorporated into its genome. In an even more particular embodiment, the PARC transgene is expressed in the respiratory tissue of the transgenic animal.

The cells may also be modified prior to their use in the screening methods described herein. For example, the cells may comprise genetic constructs designed to elucidate differences in the tested activity, e.g., PARC activity, PKCα activity and Sp1 activity, within the cell. In one assay format, cell lines that contain reporter gene fusions between the open reading frame and any assayable fusion partner may be prepared. Numerous assayable fusion partners are known and readily available including the firefly luciferase gene and the gene encoding chloramphenicol acetyltransferase (Alam et al., 1990 Anal. Biochem. 188: 245-254). Cell lines containing the reporter gene fusions are then exposed to the test substance under appropriate conditions and time. Differential expression of the reporter gene between samples exposed to test substance and control samples identifies substances that can modulate the expression of a nucleic acid encoding PARC, or an upstream activator molecule or a downstream effector molecule.

Additional examples of manipulated cells that may be used for screening methods include, but are not limited to, cells that have been transfected, transformed or infected with genetic constructs comprising PARC and/or one of its effector molecules. For example, recombinant replication-deficient adenovirus comprising the PARC gene may be operably linked to a promoter within the framework of the viral genome. Cultured cells or in vivo cells may then be infected with adenovirus and used to screen substances for their ability to inhibit the activity of PARC or one of its downstream effector molecules. An example of a genetic construct used to manipulate cells for use in the methods herein includes, but is not limited to, AdV-PARC. Itratracheal instillation of the AdV-PARC in mice causes severe perivascular and peribronchial accumulation of mononuclear cells, in particular, cells that are CD3+, CD4+ or CD8+, and B220+. Within these infiltrates of T cells, collagen accumulation is detected by Trichrome staining. Infection of mice with AdV-PARC also causes severe medial thickening of pulmonary vasculature. Mice treated with anti-lymphocyte serum prior to infection with AdV-PARC do not exhibit pulmonary accumulation of mononuclear cells.

Additional assay formats may be used to monitor the ability of the substance to modulate the expression of a nucleic acid encoding a protein such as a PARC, or an upstream or downstream signaling protein. For instance, mRNA expression may be monitored directly by hybridization to the nucleic acids of the invention. Cell lines are exposed to the substance to be tested under appropriate conditions and time and total RNA or mRNA can be isolated by standard procedures such those disclosed in Sambrook et al. (1989). Probes to detect differences in RNA expression levels between cells exposed to the agent and control cells may be prepared from the nucleic acids of the invention. Probes may be designed from the nucleic acids of the invention through methods known in the art. For instance, the G+C content of the probe and the probe length can affect probe binding to its target sequence. Methods to optimize probe specificity are commonly available in Sambrook et al. (1989) or Ausubel et al. (Current Protocols in Molecular Biology, Greene Publishing Co., New York, 1995). Probes may be designed to hybridize selectively with target nucleic acids under conditions that maximize the difference in stability between the probe:target hybrid and potential probe:non target hybrids, such as high stringency conditions, methods of which are well known in the art.

Hybridization conditions are modified using known methods, such as those described by Sambrook et al. (1989) and Ausubel et al. (1995) as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a porous glass wafer. The glass or silica wafer can then be exposed to total cellular RNA or polyA RNA from a sample under conditions in which the affixed sequences will specifically hybridize. Such glass wafers and hybridization methods are widely available, for example, those disclosed by Beattie (WO 95/11755). By examining for the ability of a given probe to specifically hybridize to an RNA sample from an untreated cell population and from a cell population exposed to the agent, agents can be assayed for their ability to up or down regulate the expression of a nucleic acid encoding the PARC, or an upstream or downstream signaling protein.

The present invention also relates to methods of screening and/or identifying compounds useful for treating, preventing or preventing the progression of fibrosis, comprising identifying substances that inhibit the activity of PKCα. Specifically, the methods of identifying such inhibitory substances comprise (a) providing a test substance to a cell, wherein the cell possesses PKCα activity, (b) measuring the amount of PKCα activity in the test cell; and (c) comparing the amount of PKCα activity in a control cell not treated with the test substance, wherein a decreased amount PKCα activity in the test cell, as compared to the control cell, indicates that the test substance inhibits PKCα activity, and may thus be used for treating, preventing or preventing the progression of fibrosis.

As used herein, “PKCα activity” is assessed by direct or indirect means. For example, PKCα activity can be directly assessed by measuring or quantifying levels of PKCα protein that binds to a receptor for C kinases (RACK), or quantifying the levels of PKCα that are produced by a cell, or measuring translocation activity of PKCα; and PKCα activity can be indirectly assessed by measuring or quantifying a detectable effect that PKCα protein has on a cell. Detectable effects that PKCα has on a cell encompass RNA transcription, protein expression or secretion, such as, but not limited to, collagen expression, or the generation of second messenger or intracellular signals. For example, PKCα activity can be assessed by such means that include, but are not limited to, phosphorylation of second messenger molecules, such as ERK1 and ERK2 and other protein kinases, generation of other second messenger signals such as Ca⁺² release, calmodulin binding, inositol triphosphatase activity, GTPase activating protein (GAP) activity and adenylate cyclase activity to name a few. Other indirect measures of PKCα activity include activation of transcription factors, such as, but not limited to Sp1, Smad3 and Smad4 to name a few, and levels of mRNA of specific transcripts.

The scope of the invention is not limited to means of measuring PKCα activity for the purposes of comparing test substances. Thus, in one embodiment, the present invention provides methods of identifying inhibitory substances of PKCα, with the methods comprising (a) providing a test substance to a cell, wherein the cell possesses PKCα activity as measured by a means of measuring PKCα activity, and (b) comparing the amount of PKCα activity, as assessed by the measuring means, in a control cell to which has not been provided the test substance, with the amount of PKCα activity in the test cell, wherein a decrease in the amount PKCα activity in the test cell, compared to the amount of PKCα activity in the control cell, indicates that the test substance inhibits PKCα activity. The measuring means may be directly correlative or inversely correlative, so long as the measuring means provides the technician with a level of PKCα activity in a cell that can be compared to other levels of PKCα activity in different cells.

The present invention also relates to methods of screening and/or identifying compounds useful for treating, preventing or preventing the progression of fibrosis, comprising identifying substances that inhibit the activity of Sp1. Specifically, the methods of identifying such inhibitory substances comprise (a) providing a test substance to a cell, wherein the cell possesses Sp1 activity, (b) measuring the amount of Sp1 activity in the test cell; and (c) comparing the amount of Sp1 activity in a control cell not treated with the test substance, wherein a decreased amount Sp1 activity in the test cell, as compared to the control cell, indicates that the test substance inhibits Sp1 activity, and may thus be used for treating, preventing or preventing the progression of fibrosis.

As used herein, “Sp1 activity” is assessed by direct or indirect means. For example, Sp1 activity can be directly assessed by measuring or quantifying levels of Sp1 protein that binds to DNA, or quantifying the levels of Sp1 that are produced by a cell, or measuring the phosphorylation Sp1; and Sp1 activity can be indirectly assessed by measuring or quantifying a detectable effect that Sp1 protein has on a cell. Detectable effects that Sp1 has on a cell encompass RNA transcription, protein expression or secretion, such as, but not limited to, collagen expression. Other indirect measures of Sp1 activity include activation or inhibition of other transcription factors, such as, but not limited to Smad3 and Smad4 to name a few

The scope of the invention is not limited to means of measuring Sp1 activity for the purposes of comparing test substances. Thus, in one embodiment, the present invention provides methods of identifying inhibitory substances of Sp1, with the methods comprising (a) providing a test substance to a cell, wherein the cell possesses Sp1 activity as measured by a means of measuring the Sp1 activity, and (b) comparing the amount of Sp1 activity, as assessed by the measuring means, in a control cell to which has not been provided the test substance, with the amount of Sp1 activity in the test cell, wherein a decrease in the amount Sp1 activity in the test cell, compared to the amount of Sp1 activity in the control cell, indicates that the test substance inhibits Sp1 activity. The measuring means may be directly correlative or inversely correlative, so long as the measuring means provides the technician with a level of Sp1 activity in a cell that can be compared to other levels of Sp1 activity in different cells.

The examples below are illustrative and not intended to limit the scope of the invention described herein.

### EXAMPLES

### Example 1

Primary Fibroblast Cell Culture. Four normal adult primary lung fibroblast cultures (LF1-LF4) were purchased from Cambrex (Walkersville, Md.). Fibroblast cultures were maintained in T75 culture flasks in humidified atmosphere of 5% CO2 at 37° C. in high-serum tissue culture medium, which was Dulbecco's modified Eagle's medium supplemented with 10% bovine calf serum, 2 mM glutamine, 2 mM sodium pyruvate, and 50 mg/liter gentamicin (all from Life Technologies, Grand Island, N.Y). Before experiments, cell cultures were preincubated for 24 h in similar conditions, except that low-serum (0.5% dialyzed bovine calf serum with no TGF-β detectable by enzyme-linked immunosorbent assay [ELISA] as described below) medium was used, supplemented in addition to the mentioned reagents with 0.28mM ascorbic acid and 0.2 mM β-aminopropionitrile (Sigma, St. Louis, Mo.). Cell culture medium for all experiments was the same low-serum medium. In all experiments, fibroblast cell cultures were tested in passages three to eight.

Recombinant Human Cytokines, Anti-Cytokine Antibodies, and Other Reagents. Recombinant human (rh) PARC, TGF-β1, TGF-β2, and TGF-β3, were purchased from R&D Systems (Minneapolis, Minn.). Carrier-free rhPARC was purchased from Cell Sciences (Norwood, Mass.). PARC was used to stimulate collagen production in fibroblast cultures at 300 ng/ml. TGF-β1 was used as a positive control for fibroblast stimulation at 1 ng/ml. Neutralizing anti-human PARC and pan-TGF-β (monoclonal mouse IgG1 clone 1D11 and purified rabbit polyclonal IgG) antibodies were purchased from R&D Systems and used for neutralization experiments in effect-saturating concentrations of 1 μg/ml and 100 μg/ml, respectively. Latency-associated peptide (rhLAP, R&D Systems) was used for TGF-β neutralization at 400 ng/ml, and aprotinin was used to inhibit TGF-β activation (Lee C G et al.) at the highest recommended by the supplier (Calbiochem, La Jolla, Calif.) concentration of 2 μg/ml.

Collagen Production Assays. Collagen was quantified as described elsewhere (Atamas S P et al.). Briefly, fibroblasts were cultured with β-aminopropionitrile (Sigma) to prevent collagen cross-linking, and Western blotting assays for collagen were performed using rabbit affinity purified anti-collagen type I antibody (Rockland, Gilbertsville, Pa.). Before electrophoresis, samples were reduced and denatured by boiling in Laemmli buffer containing β-mercaptoethanol. Human purified collagen type I (Southern Biotech, Birmingham, Ala.) was used as a positive control in these assays. The identity of collagen bands was confirmed by sensitivity to pepsin and collagenase (both from Sigma) digestion, as described (Atamas S P et al.). Under these conditions, pro-collagen appears as a single or double band around 175 kDa (Gaidarova S et al., Stefanovic B et al.). Images were collected using a Storm densitometer and band densities analyzed with ImageQuant software (Molecular Dynamics, Sunnyvale, Calif.).

Transforming Growth Factor-β ELISA Assays. ELISA kits for TGF-β1 and TGF-β2 were purchased from R&D Systems. ELISA for TGF-β3 was custom developed using a DuoSet ELISA Development System (R&D Systems) following manufacturer's recommendations. Fibroblast culture supernatants and whole cell lysates after 3, 6, 12, 24, and 48 hrs incubation were activated by acidification prior to the assay to quantify total (active and latent) TGF-β or assayed without acidification to quantify active TGF-β. Recombinant human TGF-β1, TGF-β2, and TGF-β3 from R&D Systems were used in serial dilutions for calibration of the assays. The minimal detection level in these assays was 10-20 pg/ml of TGF-β. Low serum cell culture medium containing 0.5% dialyzed fetal bovine serum had no detectable TGF-β and was used as a negative control in these assays. Each of LF1-LF4 cultures was tested in duplicates for each time point in at least three independent experiments.

Real-time PCR Quantification of Steady-State mRNA Levels. Total RNA was purified from fibroblast cultures using Trizol reagent (Invitrogen, Carlsbad, Calif.) following the manufacturer's recommendations. First-strand cDNA was synthesized in a 20-μl reaction mixture containing 1 μg of total RNA, 50 mM Tris HCl (pH 8.3), 75 mM KCl, 3 mM MgCl2, 0.5 mM of each dNTP, 1 μM of random hexamer primers, and 400 units of Moloney murine leukemia virus RT (Invitrogen). The reaction mixture was incubated at 37° C. for 60 minutes, then heated at 95° C. for 5 minutes. Real-time PCR was performed on a LightCycler system (Roche, Indianapolis, Ind.). The primers, PCR protocol, and product quantification for the internal control 18S ribosomal RNA were exactly as reported previously (Schmittgen T D et al.). All other primers and hybridization probes were designed and prepared by TIB Molbiol (Adelphia, N.J.). The hybridization probes were labeled with fluorescein at the 3′-terminus (3FL) of one probe and with LightCycler Red at the 5′-terminus (5LC) of the other probe. Amplification of a single PCR product was confirmed by gel electrophoresis and melting curve analyses. Sequences of primers and hybridization probes are provided in Table 1. The PCR reaction mixture included 5 mM MgCl2, 0.5 μM primers, 0.2 μM probes, and the recommended components of the FastStart DNA Master Hybridization Probes hot start reaction mix (Roche). The following PCR conditions were used: activation at 95° C. for 10 min, followed by 35 cycles of denaturation at 95° C. for 3 s, primer annealing at 50° C. for 5 s, fluorescence readout (F12/F11) at 50° C., and extension at 72° C. for 15 s. Fold difference in gene expression relative to 18S rRNA between treated and untreated cultures was calculated using the 2-ΔΔCT method (Livak K J et al.).

Mink Lung Epithelial Cell Proliferation Assay for Active TGF-β. Mink lung epithelial cell line CCL-64 was purchased from American Type Culture Collection (ATCC, Manassas, Va.) and maintained in high-serum medium. For proliferation assays, these cells were plated in 96-well plates at 1×104 cells/well in low-serum fibroblast cell culture medium and incubated overnight in humidified atmosphere of 5% CO2 at 37° C. The medium was then replaced with fibroblast cell culture supernatants or titrated dilutions rhTGF-β in low-serum medium, with and without neutralizing anti-pan-TGF-β antibody (clone 1D11 from R&D Systems), and incubation continued for additional 24 hours. Six hours before the end of the assay, wells were pulsed with 1 μCi of 3H-thymidine (Amersham). Quadruplicate cultures were harvested automatically and tritiated thymidine incorporation was measured with a TopCount NXT liquid scintillation counter (Perkin Elmer, Downers Grove, Ill.). Data were expressed as percent inhibition (versus low-serum medium used as a negative control), neutralized by 1 μg/ml of anti-TGF-β antibody (this concentration of the antibody was established in preliminary experiments as sufficient to completely abrogate the inhibitory effect of the fibroblast conditioned medium on mink lung epithelial cell proliferation).

Statistical Analyses. Depending on the assay, cultures were tested in duplicate, triplicate, or quadruplicate in three independent experiments for LF1 -LF4. Data were expressed as mean value ±standard deviation. The significance of differences was analyzed using two-tailed unequal variance Student's t-test or one-way ANOVA with post hoc (Scheffe) testing. A probability value (p) less than 0.05 was considered statistically significant. Statistical analyses were performed using Statistica software (StatSoft, Tulsa, Okla.).

### Example 2

### Effect of Fibroblast Stimulation With rhPARC On the Levels of Total And Active Autocrine TGF-β

To determine whether stimulation of primary lung fibroblast cultures with PARC causes an increase in autocrine TGF-β, ELISA assays of fibroblast cell culture supernatants for total (active and latent) and active TGF-β1, TGF-β2, and TGF-β3 were performed. No total or active TGF-β2 or TGF-β3, and no active TGF-β1 were detected in these assays in any of the studies culture supernatants. However, total TGF-β1 was decreased after stimulation of cultures with PARC in a time-dependent fashion (FIG. 13A,B). The decrease was significant (p<0.05, one-way ANOVA with post hoc testing) in all cases except after 3 hours of activation in some cultures and after 48 hrs in LF2 (FIG. 13B).

Real-time PCR assays were used to quantify changes in steady-state levels of TGF-β1, TGF-β2, TGF-β3, and collagen α2(I) mRNA against levels of 18S rRNA which was used as a reference. No difference in steady-state levels of TGF-β mRNAs between PARC-treated and control cultures was found (fold difference versus control at 90 minutes is shown for TGF-β1 mRNA in LF1 and LF4 in panels 1C and 1D respectively). Levels of TGF-β2 and TGF-β3 mRNAs were significantly lower and did not change after stimulation of cultures with PARC. At the same time, levels of collagen α2(I) mRNA changed in a time-dependent fashion, although some heterogeneity among individual cultures in the amplitude and kinetics of the response was observed (FIG. 13C,D).

Mink lung epithelial cell proliferation assays (FIG. 13E,F) showed that conditioned media from fibroblast cultures inhibited thymidine incorporation, and this inhibition could be reversed by anti-TGF-β-specific antibody (FIG. 13E). These observations suggested that active TGF-β, although not detected by ELISA, was present in the studied cultures. Conditioned media from the PARC-treated fibroblast cultures inhibited mink cell proliferation more than media from control non-treated fibroblasts (FIG. 13E,F). The difference between PARC-treated and control cultures in their ability to inhibit mink cell proliferation was much smaller than the ability of both types of the conditioned media to inhibit mink cell proliferation in comparison to cell culture medium (FIG. 13E).

Example 3

### Effect of TGF-β Neutralization On PARC-Stimulated Collagen Production

All of the tested fibroblast cultures responded to PARC stimulation by increasing collagen production (FIG. 14). Neutralization of autocrine TGF-β with anti-pan-TGF-β monoclonal (FIG. 14B) and polyclonal antibodies inhibited stimulating effect of TGF-β but not the effect of PARC (FIG. 14A) on collagen production by fibroblast cultures. Recombinant latency-associated peptide also inhibited the effect of TGF-β (FIG. 14D) but not the effect of PARC (FIG. 14C). An inhibitor of proteases Aprotinin that is known for its ability to inhibit TGF-β activation (Lee C G et al.) also failed to inhibit the effect of PARC on collagen production by fibroblast cultures (FIG. 14E). Aprotinin was not tested with rhTGF-β stimulation as this cytokine is already supplied in the active form. Experiments with neutralizing anti-TGF-β antibodies were repeated in LF1, LF3, and LF4, on at least two occasions in each of these cultures, with consistent results. Neutralizing experiments with rhLAP and aprotinin were repeated at least twice in LF1 and LF4, with consistent results. Densities of the scanned bands corrected for the gel background were measured from the gel images and were significantly reduced in the samples represented by the lane 3 compared to lane 2 in panels B and D (p<0.05) but not in panels A, C, and E (p>0.05) in FIG. 14.

### Example 4

Recombinant human (rh) PARC and rhlL-4 were purchased from R&D Systems (Minneapolis, Minn.). Carrier-free rhPARC was purchased from Cell Sciences (Norwood, Mass.). Neutralizing antihuman PARC antibody was purchased from R&D Systems.

Fibroblast Cell Lines. Four normal human lung fibroblast lines (LF1-LF4) derived from primary lung explants from adult donors were purchased from Bio-Whittaker (Walkersville, Md.). Three normal adult human dermal fibroblast lines (DF1-DF3) were previously established in the laboratory from primary dermal explants, as described (10). Fibroblast lines were maintained in T75 culture flasks in humidified atmosphere of 5% CO2 at 37° C. in high-serum tissue culture medium, which was Dulbecco's modified Eagle's medium supplemented with 10% bovine calf serum, 2 mM glutamine, 2 mM sodium pyruvate, and 50 mg/liter gentamicin (all from Life Technologies, Grand Island, N.Y.). Before experiments, cell cultures were preincubated for 24 h in similar conditions, except that low-serum (0.5% dialyzed bovine calf serum with no TGF-β detectable by enzyme-linked immunosorbent assay [ELISA] as described below) medium was used, supplemented in addition to the mentioned reagents with 0.28 mM ascorbic acid and 0.2 mM β-aminopropionitrile (Sigma, St. Louis, Mo.). Cell culture medium for all experiments was the same low-serum medium. In all experiments, fibroblast cell lines were tested in passages three to seven.

Real-Time Polymerase Chain Reaction for Collagen mRNA. Total RNA was purified from fibroblast monolayers using Trizol (Gibco Invitrogen, Carlsbad, Calif.), as described. Collagen mRNA was measured by real-time polymerase chain reaction (PCR) (LightCycler; Roche, Indianapolis, Ind.). The primers and hybridization probes for α2(I) collagen mRNA and reference sequence 18S rRNA were prepared by TIB Molbiol (Adelphia, N.J.). The primers for α2(I) collagen were: forward, 5′-GAT GGT GAA GAT GGT CCC ACA GG-3′, (SEQ ID NO:1) and reverse, 5′-GGT CGT CCG GGT TTT CCA GGG T-3′ (SEQ ID NO:2). The hybridization probes for α2(I) collagen were labeled with fluorescein at the 3′-terminus (3FL) of one probe and with LightCycler Red (5LC) at the 5′-terminus of the other probe. The probes were: 3FL 5′-TTC CAA GGA CCT GCT GGT GAG CCT-3′ FL (SEQ ID NO:3) and 5LC 5′-TGA ACC TGG TCA AAC TGG TCC TGC AG-3′ (SEQ ID NO:4). The PCR reaction mixture included 5 mM MgCl2, 0.5 μM primers, 0.2 μM probes, and the recommended components of the FastStart DNA Master Hybridization Probes hot start reaction mix (Roche). The following PCR conditions were used for α2(I) collagen: activation at 95° C. for 10 min, followed by 35 cycles of denaturation at 95° C. for 3 s, primer annealing at 61° C. for 5 s, fluorescence readout (F12/F11) at 68° C., and extension at 72° C. for 15 s. Primers, PCR protocol, and product quantification for 18S rRNA were exactly as reported previously. The amount of α2(I) collagen mRNA was expressed relative to the amount of 18S rRNA in the same sample. Actinomycin D (transcription inhibitor) was purchased from Sigma. Cultures were tested in duplicate in three independent experiments for reach cell line, and data were analyzed using one-way ANOVA with post hoc (Scheffe) testing utilizing Statistica software (StatSoft, Tulsa, Okla.).

Collagen Production Assays. Production of collagen was assessed using metabolic labeling with ¹⁴C-proline and Western blotting with anti-collagen type I antibody. For metabolic labeling of collagen, fibroblasts were plated at 2×105 cells/well in 6-well plates (Costar, Cambridge, Mass.), in duplicates, incubated overnight in 3 ml/well of high serum medium, and then for 24 h in low-serum medium. After that, the culture medium was replaced with 1 ml/well of fresh low serum medium with or without added test substances and containing ¹⁴C-proline at 1 μCi/ml (Amersham Pharmacia Biotech). After incubation for the desired periods of time, the cell culture supernatants were collected, rapidly frozen in liquid nitrogen and freeze-dried at −70° C. The pellets were dissolved in 100 μl of reducing Laemmli buffer per 1 ml of the cell culture supernatant, and the samples were electrophoretically separated in 7.5% acrylamide gels. Alternatively, samples were concentrated 10-fold by filter centrifugation on 30K Ultrafree-MC filters (Millipore, Bedford, Mass.). Fluorographic images were developed using EN[3H]ANCE autoradiography enhancer (NEN, Boston, Mass.). Gel images were acquired using Storm densitometer (Molecular Dynamics, Sunnyvale, Calif.), and the densities of the bands were analyzed with ImageQuant software (Molecular Dynamics). Dependence of collagen production by fibroblast cultures on the dose of PARC and duration of stimulation were evaluated using one-way ANOVA with post hoc testing.

Western blotting assays for collagen were performed using rabbit affinity purified anti-collagen type I antibody (Rockland, Gilbertsville, Pa.). Before electrophoresis, samples were reduced and denatured by boiling in Laemmli buffer containing β-mercaptoethanol. Human purified collagen type I (Southern Biotech, Birmingham, Ala.) was used as a positive control in these assays.

The identity of collagen bands was confirmed by sensitivity to pepsin and collagenase (both from Sigma) digestion. For pepsin digestion, 2 μl of 1 Macetic acid were added to 40 μl of concentrated fibroblast culture supernatant, followed by 1 μl of pepsin stock, to achieve final concentration of pepsin as indicated in the text. After 15 min of digestion at room temperature, reaction was stopped with 4 μl of 1 M Tris base and 40 μl of reducing Laemmli buffer. For collagenase digestion, 1 μl of 4 U/ml bacterial collagenase and 1 μl of protease inhibitor cocktail (Sigma) were added to 40 μl of sample and digestion performed at room temperature for 30 min. The bacterial collagenase was highly purified and had minimal clostripain and neutral protease activity, as tested by the manufacturer, according to the product information sheet.

Cell Proliferation Assays. For CellTiter AQueous 96 Non-Radioactive Cell Proliferation Assay (Promega, Madison, Wis.), fibroblasts were plated in low-serum medium at 2×103 cells/well in 96-well flat-bottom tissue culture plates (Costar) in 0.2-ml cultures and stimulated with increasing concentrations of rhPARC in quadruplicates. Low-serum tissue culture medium alone was the negative control. The Cell-Titer proliferation assay was performed according to the manufacturer's instructions, after the fibroblasts were incubated with test substances for 3-8 d. Data were expressed as mean OD490±SD of quadruplicate cultures and analyzed using one-way ANOVA with post hoc testing.

Immunoblotting for Phosphorylation of EKR1/2 and p38. Fibroblasts were plated in 6-well plates (Costar) at 2×105 cells/well in 3-ml cultures. After incubation with PARC for 15 min, fibroblast cultures were washed with ice-cold phosphate-buffered saline containing 100 μM Na3 VO4. Then, fibroblasts were lysed with 250 μl of Laemmli sample buffer. Electrophoretic separation of cell lysates was done in 7.5% acrylamide gels, and bands were transferred onto Immobilon NC membranes (Millipore, Bedford, Mass.). Membranes were probed with specific primary antibodies at 1/200 dilution, then secondary goat anti-mouse IgG-HRP conjugate (Upstate, Lake Placid, N.Y.), and visualized with an ECL detection system (Pierce, Rockford, Ill.) that was used according to the manufacturer's directions. Anti-ERK, anti-P38, anti-phospho-ERK1/2 and anti-phospho-p38 mAb were purchased from Upstate.

Gel images were collected using a Storm densitometer and band densities analyzed with ImageQuant software (Molecular Dynamics). ERK inhibitor PD98059 and p38 inhibitor SB203580 purchased from Upstate were >98% chromatographically pure and quality control tested by the supplier, and confirmed to selectively inhibit their target enzymes. Cell viability in the presence of inhibitors was determined using Trypan Blue exclusion assays.

Inhibition of Receptor Signaling with Bordetella Pertussis Toxin. Wild-type pertussis toxin (PT) and inactive mutant PT (PT9K/129G) were purified from Bordetella pertussis W28 culture supernatant by fetuin affinity chromatography as previously described. Both wild-type and inactive mutant PT were added to fibroblast cell cultures in final concentration of 10 ng/ml, fibroblasts were stimulated with rhPARC, and ERK1/2 phosphorylation tested by Western blotting after 15 min of activation with PARC.

Transforming Growth Factor-β1 ELISA Assays. ELISA kits for transforming growth factor (TGF)-β1 were purchased from R&D Systems (Minneapolis, Minn.), and fibroblast culture supernatants and whole cell lysates after 3, 6, 12, 24, 48, and 72 h activation with rhPARC were assayed in duplicates for total TGF-β1, according to the manufacturer's instructions. Low-serum cell culture medium containing 0.5% dialyzed fetal bovine serum had no detectable TGF-β1 and was used as a negative control in these assays.

### Example 5

### Production of Type I Collagen Protein Is Increased In Response To PARC

To test effects of rhPARC on collagen protein production by lung and dermal fibroblast cell lines, fibroblast cultures were incubated for 48 h with or without 30 ng/ml and 300 ng/ml rhPARC in the low-serum cell culture medium containing 14Cproline for metabolic labeling of collagen. Fibroblast culture supernatants contained two 14C-proline containing bands in gel electrophoresis under reducing conditions (FIGS. 13A and 13B). Digestion with chromatographically pure bacterial collagenase in the presence of protease inhibitors eliminated the bands completely (FIG. 13A). To confirm the identity of these bands, we conducted Western blotting experiments in reducing conditions of fibroblast culture supernatants using an anti-collagen type I antibody, that recognizes α1(I) collagen but not other collagen chains. Digestion with pepsin caused expected decrease in the apparent molecular weight of the immunoreactive collagen from ˜175 kD to ˜125 kD (FIG. 13C).

Exposure to rhPARC from both sources (R&D Systems and Cell Sciences) for 48 h increased fibroblast production of collagen more than 3-fold over control in five out of seven tested fibroblast lines (FIG. 13). The stimulating effect of rhPARC on collagen production was inhibited by adding 100 μg/ml of neutralizing anti-PARC antibody to the cell cultures (FIG. 13D). Three lung fibroblast lines and two dermal lines, each tested in at least three experiments, all responded to rhPARC stimulation, with a dose of 300 ng/ml consistently stimulating collagen production. The average increase in the density of collagen bands after 48 h of stimulation with 300 ng/ml rhPARC was 3.4±0.9 fold (P<0.01, Student's t test, compared with control unstimulated cultures). One lung fibroblast line and one dermal fibroblast line were consistently non-responsive to rhPARC stimulation up to 72 h and rhPARC concentrations up to 1,000 ng/ml.

Further experiments defined the dose-response (FIG. 14A) and the kinetics of PARC's effect (FIG. 14B) on collagen production in LF1 and LF4 lines, with a total of five experiments done with similar results. One-way ANOVA analyses with post hoc testing revealed a significant increase in collagen production in response to 30 ng/ml (P<0.05) and 300 ng/ml (P<0.01) rhPARC, with increases also seen at 3 ng/ml (P<0.12) and 10 ng/ml (P<0.1) rhPARC. There was no difference in collagen production between control cultures and those exposed to PARC for 3, 6, and 12 h of activation, with increase in collagen production not exceeding 1.2±0.2-fold over control non-treated cells (P>0.05). A significant increase in collagen production (P<0.05) was observed after 24, 48, and 72 h of activation with rhPARC (FIG. 14B).

### Example 6

### Collagen α2(I) mRNA Is Increased In Response To PARC

Experiments were done to test whether an increase in steady-state collagen mRNA levels might be a mechanism of PARC stimulation of collagen protein production. Fibroblast lines LF1 and LF2 were incubated in the low-serum cell culture medium with and without 300 ng/ml rhPARC for 0 min, 90 min, 3 h, 6 h, and 24 h. Total mRNA was purified and collagen mRNA levels were tested in a semiquantitative manner.

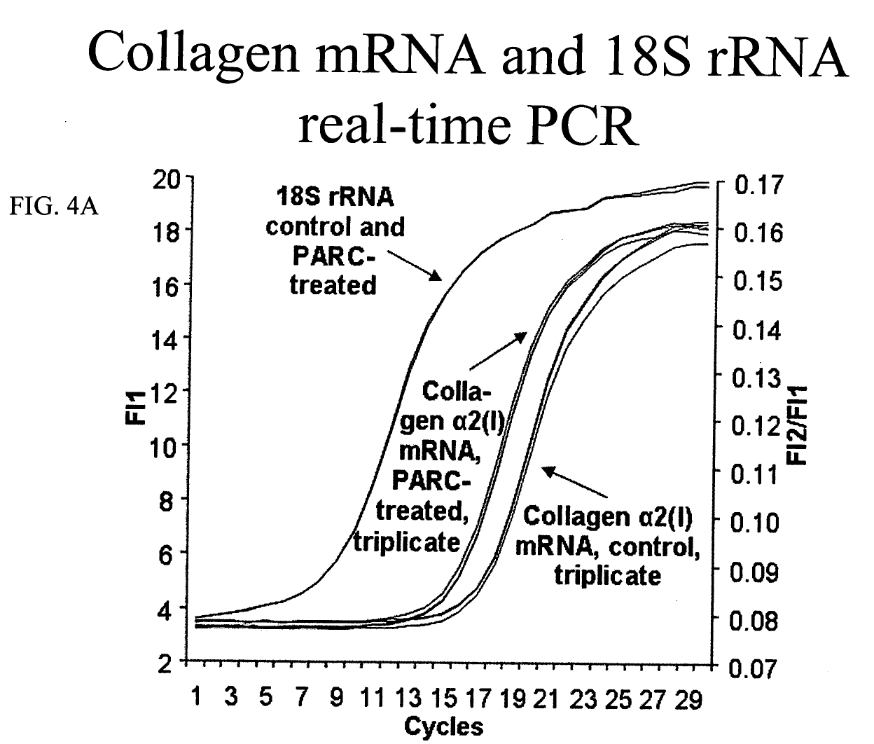

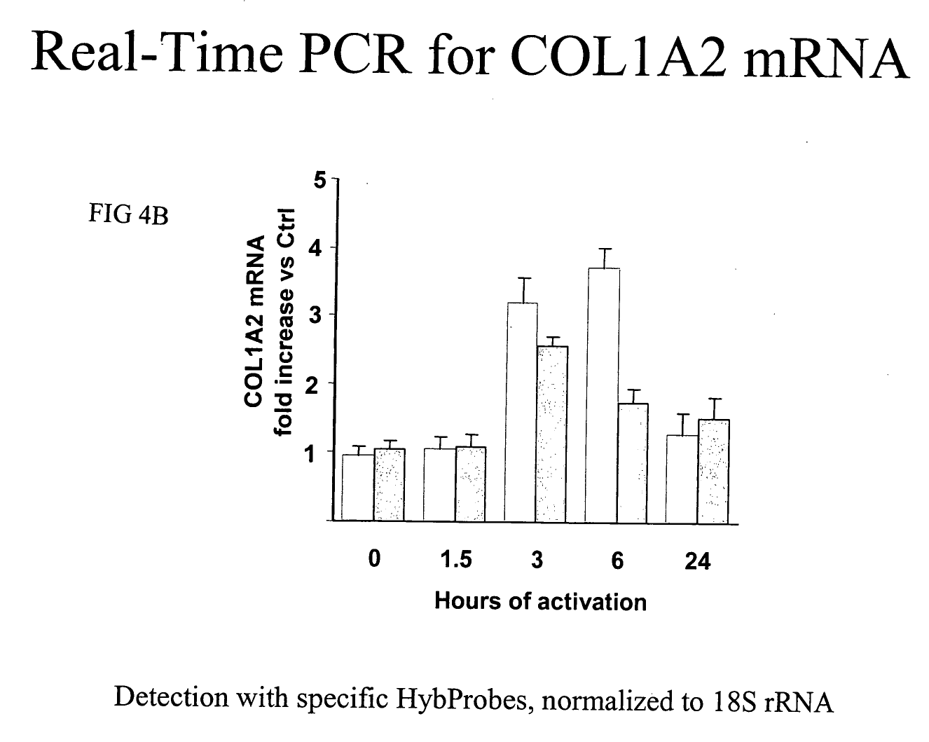

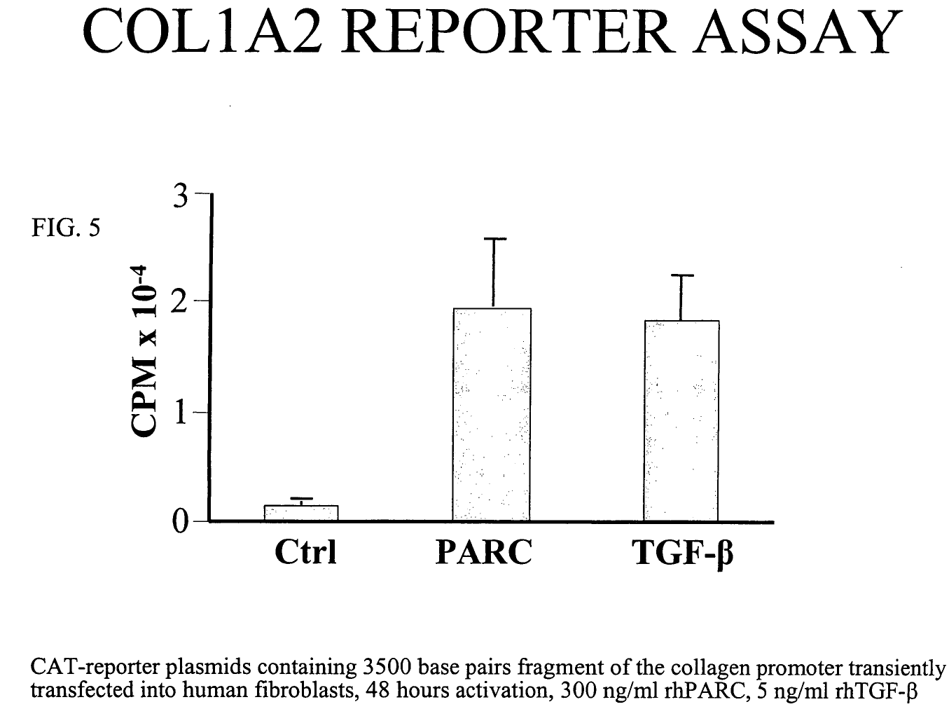

RT-PCR with levels of collagen mRNA determined relative to 18S rRNA levels. The steady state mRNA levels for α2(I) collagen chain were transiently increased between 3 and 6 h from the time of activation (FIG. 4). Treatment of cell cultures with 10 μg/ml actinomycin D (inhibitor of transcription) before stimulation with PARC completely abrogated the increase in collagen mRNA. Both fibroblast lines were tested each in three separate experiments with similar results.

### Example 7

### PARC Does Not Stimulate Production of TGF-β1 From Fibroblasts

To exclude the possibility that collagen production is activated by an increase in autocrine TGF-β1 production after PARC stimulation, levels of this cytokine were measured by ELISA in supernatants and whole cell lysates from fibroblasts exposed for 3, 6, 12, 24, 48, and 72 h to 300 ng/ml rhPARC. These experiments were repeated in all studied lung and dermal fibroblast lines. Although there was significant variability in baseline TGF-β1 production between the fibroblast cell lines, the maximal increase of 1.3±0.2-fold in production of TGFβ1 by fibroblasts (P>0.05 by Student's t test) was observed in response to PARC activation.

### Example 8

### PARC Has Limited Effect On Fibroblast Proliferation

Effects of PARC on fibroblast proliferation were less pronounced than its effects on collagen production. Fibroblast cultures were incubated with and without rhPARC for 3-8 d. rhPARC was tested in various concentrations ranging from 1-3,000 ng/ml, in quadruplicate cultures. Lung and dermal lines cell lines, LF1, LF2, DF1, and DF2 were tested in proliferation assays, each line in at least two experiments. Although PARC stimulated fibroblast proliferation in these lung and dermal fibroblast lines, the maximum increase over non-stimulated control was 20-25% in lung lines and 45-50% in dermal lines on Days 7 and 8 of incubation.

### Example 9

### PARC Signals Through ERK, But Not p38, Pathways

Experiments were done to determine whether rhPARC activated phosphorylation of the MAP kinase pathways. Fibroblasts were activated with rhPARC in low-serum cell culture medium, lysed, and phosphorylation of ERK1/2 and another MAP kinase, p38, was studied by Western blotting. Two independent experiments were done, each of which tested LF1 and LF2 cell lines. Phosphorylation of ERK1/2, but not p38, was increased in lung fibroblasts (FIG. 6). ERK pathways appear critical for the effect, because PD98059, a specific inhibitor of ERK activation, also inhibited PARC activated collagen production in lung fibroblasts, whereas SB203580, a specific inhibitor of p38 activation, had no effect (FIG. 8).

### Example 10

### The PARC Receptor Is G Protein-Coupled

The molecular identity of PARC receptor remains unknown. Experiments were done to confirm that the PARC receptor is G protein-coupled, as are other chemokine receptors. LF1 fibroblasts were activated with 300 mg/ml rhPARC for 15 min alone and in the presence of 10 ng/ml Bordetella PT, which inhibits G protein-coupled signaling, or its inactive mutant. Bordetella PT, but not the inactive mutant, inhibited phosphorylation of ERK in LF1 fibroblasts in two independent experiments (FIG. 10). The PARC receptor is G protein-coupled, similar to other CC chemokine receptors.

### Example 11

### Activation of Sp1 In Response To PARC

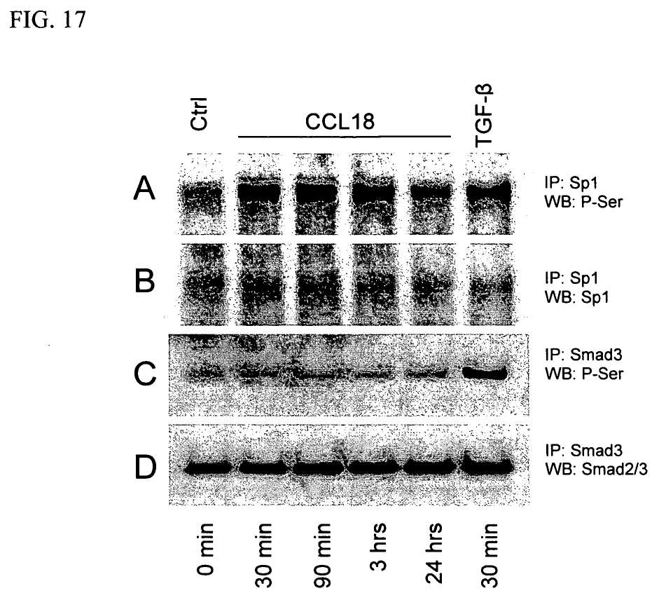

To determine if PARC stimulated phosphorylation of Sp1 and Smad3, these factors were immunoprecipitated from lysates of fibroblasts that had been activated with recombinant human PARC for up to 24 hours, and the levels of their phosphorylation were tested in Western blotting assays using anti-phosphoserine and anti-phospho-Smad2/3 antibodies. Equal loading was controlled by stripping the membranes and re-developing them with anti-Sp1, anti-Smad3 and anti-Smad2/3 antibodies (FIG. 17). Although Sp1 was phosphorylated to some extent in unstimulated control fibroblast cultures, the level of Sp1 phosphorylation was consistently increased in response to PARC stimulation in a time-dependent fashion (FIG. 17). No changes in phosphorylation of immunoprecipitated Smad3 were detected. In contrast, activation of fibroblasts with rhTGF-β1 caused increased phosphorylation of Smad3 (FIG. 17).

Preparation of fibroblast lysates, immunoprecipitation of Sp1 and Smad2/3, normalization of protein concentration in the samples with BioRad assays, electrophoretic separation, Western blotting, membrane stripping and re-development with different antibodies were performed as described in Atamas et al. (Am. J. Respir. Cell. Mol., 29:743-749 (2003); J. Immunol. 168: 1139-1145 (2002); and Arthritis Rheum., 42:1168-1178 (1999)), which are hereby incorporated by reference. Rabbit antibodies for total Smad2/3 and Sp1 were from Upstate (Waltham, Masss.). Antiphospho-Smad2/3 antibody and goat antibody for total Smad3 were from Santa Cruz Biotechnology (Santa Cruz, Calif.), as was anti-gp130 antibody used to confirm equal loading of the samples. Antiphosphoserine antibody was from Biomol (Plymouth Meeting, Pa.).

### Example 12

### DNA Binding of Sp1 In Response To PARC

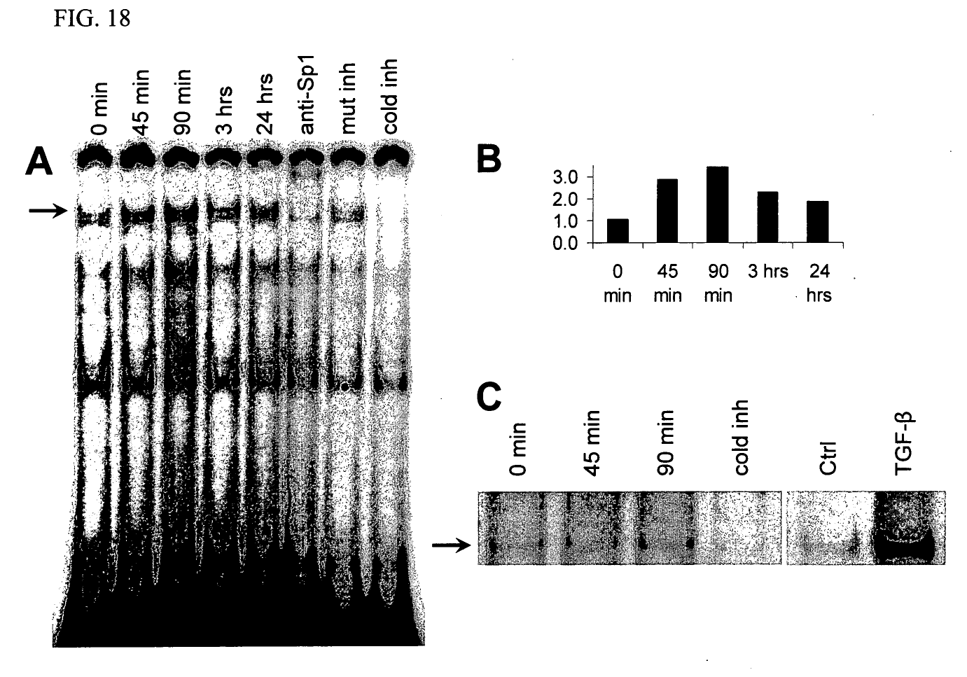

The initial comparison of DNA binding by transcription factors in nuclear lysates from PARC-activated and control fibroblasts was performed using TranSignal protein/DNA arrays, that allow for simultaneous screening of 54 different transcription factors. DNA binding by a known transcriptional activator of collagen, Sp1, was consistently increased in rhPARC-stimulated lung fibroblasts in three independent experiments with two different cell lines (LF1 and LF2, data not shown). No significant changes in DNA binding by other transcription factors were detected in these experiments (not shown). More quantitative electrophoretic mobility shift assays (EMSA) analyses revealed that PARC treatment induces a three-fold increase in DNA binding by Sp1, using both consensus and collagen α2(I) promoter-specific probes (FIG. 18A,B). No increase in DNA-binding activity of Smad3/4 occurred after activation with PARC according to EMSA analyses (FIG. 18C).

Nushif™ kits for EMSA and supershift assays and reagents for preparation of nuclear extracts were purchased from Active Motif (Carlsbad, Calif.); the procedures were performed according to manufacturer's recommendations, including end-labeling of the specific probes with [γ-³²P]ATP. In addition to the [γ-³²P]ATP-labeled Sp1 consensus probe (5′-CCCTTGGTGGGGGCGGGGCCTAAGCTGCG-3′) (SEQ ID NO:5), wild-type and mutant (5′-CCCTTGGTGGGTTGGGGGCCTAAGCTGCG-3′) (SEQ ID NO:6) oligo competitors, and an Sp1-specific antibody from the kit, an oligonucleotide representing the Sp1-binding region in the α2(I) collagen promoter (5′-CGCAGGCTCCTCCCAGCTGT-3′) (SEQ ID NO:7) was used as a probe (after end-labeling with [γ-³²P]ATP) and as a cold competitor. A corresponding mutant (5′- CGCAGGCGAATCCCAGCTGT-3′) (SEQ ID NO:8) was used as an inactive competitor. Smad3/4 consensus and mutant oligonucleotides and Smad3-specific rabbit polyclonal antibody for supershift assays were purchased from Santa Cruz Biotechnology. Nuclear extracts were adjusted for total protein content to ensure equal loading. Gels were exposed to a Phosphorimager screen and scanned using a Storm Phosphorimager (Molecular Dynamics); band densities were analyzed with ImageQuant software.

### Example 13

### Transcription Factor Sp1 Is Necessary For PARC-Induced Profibrotic Events

Transfections were performed using Metafectene (Biontex, Munich, Germany) and Mirus (Madison, Wis.) transfection reagents, using 2.5 μg of each plasmid per well in 6-well plates, following manufacturers' recommendations. Thirty-five to forty percent of fibroblasts expressed GFP at high levels by fluorescent microscopy 24 hrs after transfection or co-transfection with pEGFP-C1 vector (BD Biosciences Clontech, Palo Alto, Calif.), and further electronic accumulation of the fluorescent signal revealed that 60% to 80% of cells had higher levels of fluorescence than the control mock-transfected cells.

Collagen was quantified by Western blotting and the identity of collagen bands was confirmed by sensitivity to pepsin and collagenase digestion as described elsewhere in Atamas et al. (Am. J. Respir. Cell. Mol., 29:743-749 (2003)). In these assays, pro-collagen appears as a single or double band with a molecular weight of approximately 175 kDa (4,27,28). Selected results were confirmed using metabolic labeling of collagen with ¹⁴C-proline, followed by fluorographically enhanced autoradiography.

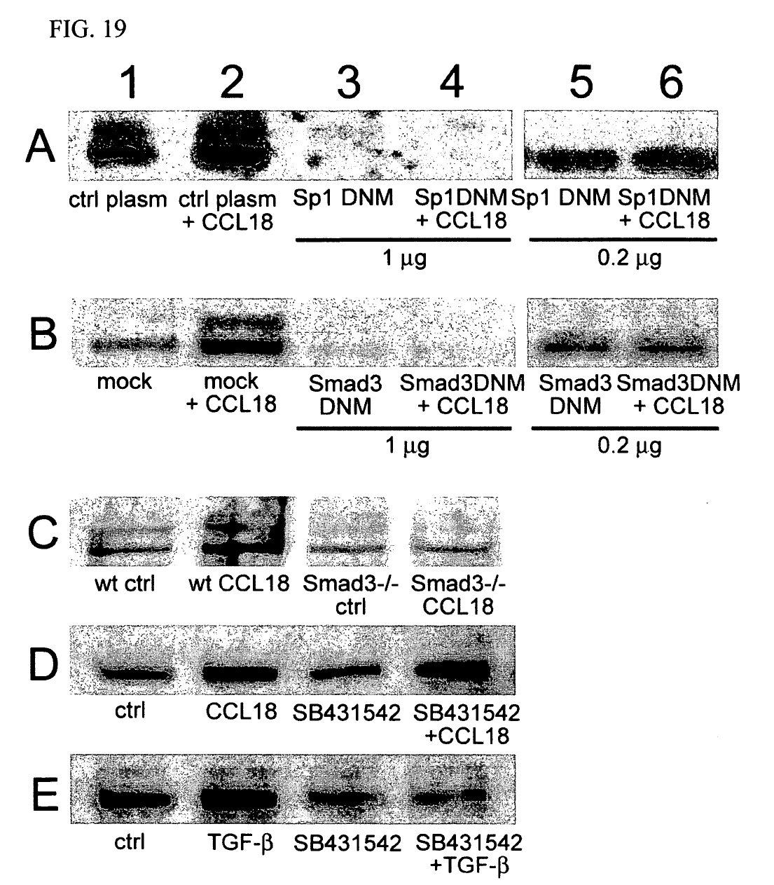

Transient transfection with 1 μg of Sp1 and Smad3 dominant negative mutant constructs abrogated basal and PARC-stimulated upregulation of collagen production, whereas transfection with 0.2 μg of the constructs inhibited the response to PARC stimulation but not basal collagen production (FIG. 19A,B). Wild-type mouse fibroblasts responded to PARC stimulation by upregulating collagen production, whereas Smad3-/-fibroblasts did not (FIG. 19C). A selective pharmacologic inhibitor of ALK5 (TGF-β receptor type 1), SB431542, blocked TGF-β-stimulated (FIG. 19E) but not PARC-stimulated (FIG. 19D) collagen upregulation, suggesting that basal activity of Smad3 is not related to TGF-β activity, but may be related to PARC's profibrotic activity.

### Example 14

### Neutralization of TGF-β Does Not Affect PARC-Induced Profibrotic Events

ELISA kits for TGF-β1, -β2, and -β3 were purchased from R&D Systems and assays performed following manufacturer's recommendations. Fibroblast culture supernatants and whole cell lysates after 3, 6, 12, 24, and 48 hrs incubation were activated by acidification prior to the assay to quantify total (active and latent) TGF-β or assayed without acidification to quantify active TGF-β. The minimal detection level in these assays was 10-20 pg/ml of TGF-β. Each culture (LF1-LF4) was tested in duplicate at each time point in at least three independent experiments. To confirm the ELISA data on active TGF-β, mink lung epithelial cell (Mv1Lu, ATCC, Manassas, Va.) proliferation assays were employed. After the fibroblast cell culture supernatants were cleared of cellular debris, ³H-thymidine incorporation was assayed. The effects of fibroblast cell culture supernatants or titrated dilutions of rhTGF-β on ³H-thymidine incorporation were tested with and without neutralizing anti-pan-TGF-β antibody (clone 1D11 from R&D Systems), following the standard protocol as described in Ghahary et al., (Wound Repair Regen. 10:328-335 (2002)), which is hereby incorporated by reference.

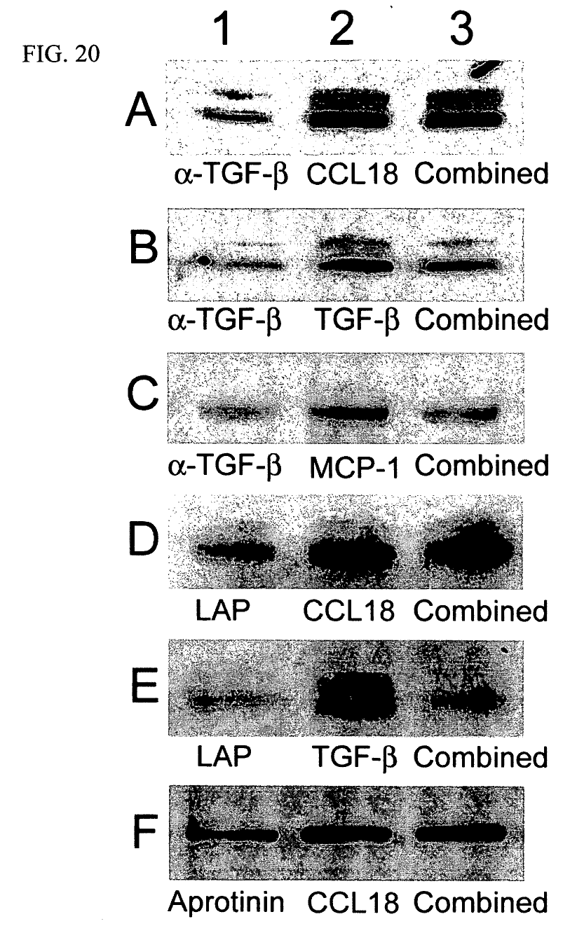

Neutralization of autocrine TGF-β, with both anti-pan-TGF-β monoclonal (FIG. 20A-C) and polyclonal (not shown) antibodies, failed to inhibit the effect of PARC on collagen production (FIG. 20A). The activation of collagen, however, expression by TGF-β or MCP-1, a chemokine known to induce collagen production in pulmonary fibroblasts through autocrine TGF-β was inhibited by treatment with anti-TGF-β antibodies (FIGS. 20B and 20C). Recombinant latency-associated peptide also inhibited the effect of TGF-β (FIG. 20E), but failed to block the effect of PARC on collagen production (FIG. 20D). Aprotinin, a protease inhibitor known for its ability to inhibit TGF-β activation, also failed to inhibit the effect of PARC on collagen production in fibroblast cultures (FIG. 20F).

What is claimed is:

1. A method of treating fibrosis in a subject in need of treatment thereof, said method comprising inhibiting the activity of a target molecule that promotes said fibrosis, with the proviso that the target molecule is not transforming growth factor-beta (TGF-β).

2. The method of claim 1, wherein said target molecule that promotes said fibrosis is pulmonary activation-regulated chemokine (PARC) in said subject.

3. The method of claim 2, wherein said fibrosis is pulmonary fibrosis.

4. The method of claim 3, wherein said pulmonary fibrosis is a symptom of a condition selected from the group consisting of scleroderma lung disease, sarcoidosis, hypersensitivity pneumonitis, rheumatoid arthritis, lupus, asbestosis and idiopathic pulmonary fibrosis.

5. The method of claim 4, wherein said inhibiting the activity of said PARC comprises inhibiting the binding of PARC to its receptor.

6. The method claim 4, wherein said inhibiting the activity of said PARC comprises inhibiting the expression of PARC.

7. The method of claim 4, wherein said inhibiting the activity of said PARC comprises inhibiting the activity of an effector molecule of PARC.

8. The method of claim 7, wherein said effector molecule is selected from the group consisting of Sp1 transcription factor and protein kinase C-alpha (PKCα).

9. The method of claim 8, wherein said effector molecule is PKCα.

10. The method of claim 9, wherein said inhibition of PKCα comprises RNA antisense inhibition.

11. The method of claim 9, wherein said inhibition of PKCα comprises a pharmaceutically effective amount of a PKCα antagonist.

12. The method of claim 11, wherein said PKCα antagonist is selected from the group consisting of diacylglycerol kinase zeta (DKGζ) and pseudosubstrate peptides.

13. The method of claim 8, wherein said effector molecule is transcription factor Sp1.

14. The method of claim 13, wherein said inhibition of Sp1 comprises RNA antisense inhibition.

15. The method of claim 13, wherein said inhibition of Sp1 comprises a pharmaceutically effective amount of a Sp1 antagonist.

16. A method of identifying compounds useful for the treatment of fibrosis, said method comprising
a) providing a test substance to a cell, wherein said cell possesses PARC activity,
b) measuring the amount of said PARC activity in said test cell; and
c) comparing the amount of PARC activity in a control cell, said control cell having not been provided said test substance, with the amount of PARC activity,
wherein a decrease in the amount of PARC activity in said test cell, compared to amount of PARC activity in said control cell indicates that said test substance is useful for treating, preventing or preventing the progression of fibrosis

17. The method of claim 16, wherein said method is performed in cell culture.

18. The method of claim 17, wherein said cell culture is derived from a transgenic animal.

19. The method of claim 18, wherein said transgenic animal is a transgenic mouse comprising at least one copy of human pulmonary and activation-regulated chemokine gene in its genome.

20. A method of identifying compounds useful for the treatment of fibrosis, said method comprising
a) providing a test substance to a cell, wherein the cell possesses PARC activity as measured by a means of measuring said PARC activity,
b) measuring the amount of PARC activity in said test cell by said means; and
c) comparing the amount of PARC activity in a control cell not treated with said test substance,
wherein a difference in the amount PARC activity in said test cell, compared to PARC activity in said control cell indicates that said test substance is useful for treating, preventing or preventing the progression of fibrosis.

21. A method of identifying compounds useful for the treatment of fibrosis, said method comprising
a) providing a test substance to a cell, wherein the cell possesses PKCα activity or Sp1 activity as measured by a means of measuring said PKCα activity or Sp1 activity,
b) measuring the amount of PKCα activity or Sp1 activity in said tested cell by said means; and
c) comparing the amount of PKCα activity or Sp1 activity in a control cell not treated with said test substance, as measured by said means,
wherein a difference in the amount PKCα activity or Sp1 activity in said test cell, compared to PKCα activity or Sp1 activity in said control cell indicates that said test substance is useful for treating, preventing or preventing the progression of fibrosis.

22. The method of claim 21, wherein said difference that indicates that said test substance is useful for treating, preventing or preventing the progression of fibrosis is a decrease in said PKCα activity or Sp1 activity.
